(19) United States
(12) Patent Application Publication
(10) Pub. No.: US 2026/0004908 A1
(43) Pub. Date: Jan. 1, 2026

(54) AUTOMATED MEDICATION AUTHENTICITY AND USAGE VERIFICATION

(71) Applicant: EMED POPULATION HEALTH, LLC, Miami, FL (US)

(72) Inventors: Tatiana Souslova, Tampa, FL (US); Gregory E. Page, Fort Lauderdale, FL (US); Michael W. Ferro, JR., Palm Beach, FL (US); Jeffrey M. Schumm, Miami, FL (US)

(21) Appl. No.: 19/249,794

(22) Filed: Jun. 25, 2025

Related U.S. Application Data
(60) Provisional application No. 63/664,529, filed on Jun. 26, 2024.

(51) Int. Cl. G16H 20/10; G06T 7/246; G06T 7/73; G06V 10/25; G06V 10/44; G06V 10/74; G06V 20/50; G06V 40/10; G16H 10/60; G16H 70/40

(52) U.S. Cl. CPC G16H 20/10; G06T 7/246; G06T 7/73; G06V 10/25; G06V 10/44; G06V 10/74; G06V 20/50; G06V 40/10; G16H 10/60; G16H 70/40; G06T 2207/10016; G06T 2207/30004; G06T 2207/30196; G06T 2207/30241; G06V 2201/03

(57) ABSTRACT

Systems and methods for remote verification of medication administration are disclosed herein. In some aspects, a system receives a request for verifying administration for a medication including an image. The system may identify regions of interest that include medication identifying features from the image. The system may compare a feature representation of each medication identifying feature to reference feature representations managed by authorized operator devices. The system may identify a matching medication and trigger an image acquisition session to obtain a time-series of images, at least one of the images comprising a patient in proximity to the medication. The system may extract patient features and a posture of the medication in relation to the patient and input the extracted data and entity-issued administration instructions into a model to obtain a set of patient-specific administration instructions.

### CROSS-REFERENCE TO RELATED APPLICATIONS

This application claims the benefit of U.S. Provisional Patent Application No. 63/664,529, entitled “AUTOMATED MEDICATION AUTHENTICITY AND USAGE VERIFICATION,” filed Jun. 26, 2024, the contents of which are incorporated by reference herein in their entirety.

### TECHNICAL FIELD

This application relates to approaches for remote verification of medication administration. Some embodiments relate to remote verification of authenticity of medications. Some embodiments relate to remote verification for the correct usage of medications.

### BACKGROUND

The increasing prevalence of at-home medication use has transformed the landscape of healthcare delivery, enabling patients to manage chronic conditions, acute illnesses, and preventive therapies outside of traditional clinical settings. This shift is driven by the growing availability of prescription and over-the-counter medications, advancements in telemedicine, and the need for convenient, patient-centered care. However, at-home medication administration introduces new challenges related to patient safety, adherence, and the risk of medication errors. Patients may lack the training to properly identify, store, and administer medications, particularly those requiring complex delivery methods such as injections or titrated dosing. Additionally, the rise of online pharmacies and direct-to-consumer distribution channels has increased the risk of patients inadvertently obtaining counterfeit or substandard medications.

### SUMMARY

Accordingly, approaches are disclosed herein for remote verification of medication administration that enables automated, patient-specific guidance and robust authentication of medications, thereby reducing the risks associated with counterfeit drugs and improper administration. In particular, techniques described herein enable a remote device to capture and transmit images related to a medication, its administration, or both, which can then be analyzed to verify medication authenticity, guide proper administration, or both. In some embodiments, verification of medication authenticity is performed in substantially real time. In some embodiments, administration guidance is provided in substantially real time, such that a subject can receive live guidance as they administer a medication. The various approaches herein can be combined or separated in a variety of manners. For example, separation between the processes of medication identification, patient monitoring, and the delivery of tailored administration instructions is possible, with different embodiments offering different functionality.

For example, a remote medication verification system in accordance with embodiments described herein can include one or more of remote devices, each configured to capture images of a medication, its packaging, and the administration process. Upon receiving a verification request comprising at least one image, the system can identify one or more regions of interest within the image, each containing a medication identifying feature (e.g., the medication itself, packaging, machine-readable code, or tamper-evident feature). The system can compare feature representations of these regions to a data store (e.g., a centrally managed database) of reference feature representations, which can be maintained by authorized operator devices. Based on this comparison, the system can identify a matching medication or determine that there is no match. In some embodiments, the system can, based on the identification, retrieve corresponding administration instructions.

In some embodiments, the system may trigger an image acquisition session at the remote device to obtain a time-series of images, for example after the medication and instructions are identified. At least one of the images can include at least a portion of the patient in proximity to the medication. The system can extract patient features and determine the posture (e.g., proximity, pose) of the medication relative to the patient using one or more of the images included in the time-series of images. These features, along with the administration instructions, can be input into a machine learning model to generate a set of patient-specific administration instructions. The system can transmit commands to the remote device to display these tailored instructions to the user.

In some embodiments, the system can trigger a second image acquisition session to obtain additional images for monitoring the trajectory of the medication towards the subject. Based on analysis of this time-series, the system can determine whether the trajectory is consistent with a predefined administration path. If the trajectory deviates from the expected path, the system can generate and transmit corrective instructions to the remote device, thereby improving the accuracy and safety of medication administration. For example, if a medication is to be injected in the thigh but the subject is placing an administration device near their abdomen or too close to their knee, the system can generate and transmit corrective instructions to guide the subject to properly position the administration device

In some embodiments, the feature representation used for medication identification can include a hash value, a vector embedding, a set of extracted visual features, or any other suitable information, either alone or in combination. The process of retrieving administration instructions may involve generating a search query with a medication identifier to access a remote database, or scraping data from a URL associated with the medication provider. The system can generate a set of administration instructions based on the retrieved or scraped data, ensuring that the guidance provided is both accurate and up-to-date.

Administration instructions can be provided by a wide variety of entities. For example, a drug manufacturer may be an entity and provide standard instructions for administration. As another example, an entity can be a physician who may provide specific instructions for particular subjects. For example, a physician may modify standard administration instructions to accommodate particularities of specific patients.

Various other aspects, features, and advantages of the system will be apparent through the detailed description and the drawings attached hereto. It is also to be understood that both the foregoing general description and the following detailed description are examples, and not restrictive of the scope of the disclosure. As used in the specification and in the claims, the singular forms of “a,” “an,” and “the” include plural referents unless the context clearly dictates otherwise. In addition, as used in the specification and the claims, the term “or” means “and/or” unless the context clearly dictates otherwise. Additionally, as used in the specification, “a portion” refers to a part of, or the entirety of (i.e., the entire portion), of a given item (e.g., data), unless the context clearly dictates otherwise.

### BRIEF DESCRIPTION OF THE DRAWINGS

Detailed descriptions of implementations of the present invention will be described and explained through the use of the accompanying drawings.

FIG. 1A is a block diagram illustrating a suitable computing environment for administration verification system using machine learning techniques, in accordance with embodiments herein.

FIG. 1B illustrates regions of interest comprising medication-identifying features in an image of the medication, in accordance with embodiments herein.

FIG. 1C illustrates an example repository storing precomputed feature representations for reference medication-identifying features, in accordance with embodiments herein.

FIG. 2 is a block diagram that illustrates an example process for verifying medication authenticity, in accordance with embodiments herein.

FIG. 3 is a block diagram that illustrates an example process for medication authenticity verification and administration verification, in accordance with embodiments herein.

FIG. 4 is a flowchart that illustrates an example process for comparing an input image to images in a reference library, in accordance with embodiments herein.

FIG. 5 is a flowchart that illustrates an example process for verifying a medication using a machine-readable code, in accordance with embodiments herein.

FIG. 6 is a flowchart that illustrates an example process for analyzing tamper-evident features, in accordance with embodiments herein.

FIG. 7 is a diagram that illustrates an example of identifying injection sites and an injection device, in accordance with embodiments herein.

FIG. 8 depicts a process for training an artificial intelligence or machine learning model according to some embodiments.

FIG. 9 illustrates a computer system on which certain embodiments can be run.

The technologies described herein will become more apparent to those skilled in the art from studying the Detailed Description in conjunction with the drawings. Embodiments or implementations describing aspects of the invention are illustrated by way of example, and the same references can indicate similar elements. While the drawings depict various embodiments for the purpose of illustration, those skilled in the art will recognize that alternative embodiments can be employed without departing from the principles of the present technologies. Accordingly, while specific embodiments are shown in the drawings, the technology is amenable to various modifications.

### DETAILED DESCRIPTION

Although several embodiments, examples, and illustrations are disclosed below, it will be understood by those of ordinary skill in the art that present disclosure's scope extends beyond the specifically disclosed embodiments, examples, and illustrations and includes other uses of the inventions and obvious modifications and equivalents thereof. Embodiments are described with reference to the accompanying figures, wherein like numerals refer to like elements throughout. The terminology used in the description presented herein is not intended to be interpreted in any limited or restrictive manner simply because it is being used in conjunction with a detailed description of certain specific embodiments. In addition, embodiments can comprise several novel features and no single feature is necessarily essential or solely responsible for its desirable attributes.

Verifying the authenticity of medications is important to ensure safety and efficacy. Counterfeit medications can contain incorrect dosages, incorrect ingredients, or even no active ingredients at all, which can pose a serious risk to patient safety or cause treatment programs to fail. For example, an individual using medications for weight loss may fail to lose significant weight if they are treated with a counterfeit medication that does not contain the correct active ingredient or that contains the active ingredient in lower than expected amounts, or the individual may experience greater, potentially serious, side effects if the actual dosage is too high or the medication contains a different active ingredient.

Various approaches exist for verifying medication authenticity, such as tamper-evident packaging, manufacturer verification tools (e.g., unique codes that can be checked with the manufacturer, etc.), and so forth. These and other features can help ensure that patients receive genuine medications, which can promote better health outcomes and reduce the risk of harm from ineffective or harmful counterfeit drugs. However, counterfeit medications can attempt to replicate these features, for example using similar packaging, similar tamper-evident features, copying codes from legitimate products, and so forth, making verification difficult. Nonetheless, counterfeit manufacturers often make small errors or produce products that appear genuine in isolation but which have differences when compared against legitimate products. While these small errors or variations may be overlooked by human inspection, they may be detected using the computer-implemented approaches described herein.

Existing solutions typically rely on manual verification, in which patients or pharmacists are tasked with verifying the authenticity of medications. However, patients may be ill-equipped to verify authenticity, lacking knowledge of what to look for to identify whether a particular medication is authentic or not. Given the plethora of pharmaceutical manufacturers and large number of available medications, even experienced pharmacists may be unsure of how to verify that drugs are authentic. Moreover, patients and pharmacists may simply assume that a drug is authentic because it was obtained from a pharmaceutical supplier (e.g., in the case of a pharmacist) or was picked up from or delivered by a pharmacy (e.g., in the case of a patient).

Counterfeit medications can be especially challenging when there is significant demand for a medication, high cost associated with a medication, etc. For example, glucagon-like peptide 1 (GLP-1) agonists have recently experienced widespread demand due to their benefits for treating type 2 diabetes and obesity. The high demand for these drugs has resulted in shortages. To ensure availability, healthcare providers and patients have turned to compounding pharmacies, unregulated companies, and scammers. In some cases, these medications may be legitimate and serve as substitutes when brand name medications are unavailable. However, in some cases, these products may be counterfeits with the wrong, too little, too much, or no active ingredient at all. Some may contain harmful ingredients. In some cases, counterfeit medications are marketed directly to consumers. For example, the U.S. Food and Drug Administration (FDA) has issued multiple warning letters to companies marketing GLP-1 agonists to the general public. The FDA has also warned of counterfeit products and products that contain potentially unsafe or ineffective ingredients. For example, the FDA warns that products containing semaglutide salts (e.g., semaglutide sodium or semaglutide acetate) have not been shown to be safe and effective.

It can also be important to ensure that medications are taken appropriately, for example in the correct amounts, at the correct times, etc. Ensuring correct administration can be especially significant where there is a higher likelihood of patient error, such as when medications need to be taken at specific times, with or without food, in certain combinations (e.g., when a patient is on multiple medications, it can be important to ensure that certain medications are or are not taken together), and so forth. Delivery technique can be significant. For example, while most patients are likely familiar with taking oral medications (though in some cases, a patient may not realize if they are supposed to chew an oral medication or swallow it whole), other delivery techniques may be less familiar to patients. For example, semaglutide and other GLP-1 inhibitors are typically delivered via subcutaneous injection. Semaglutide is typically administered once per week. Such infrequent dosing can increase the likelihood that a patient forgets to administer the medication at the correct time or makes an error carrying out the administration due to a lack of practice or familiarity with the process.

The injection process itself can carry significant challenges. For example, a subcutaneous injection may need to be delivered via the abdomen, thigh, or upper arm. Selecting an appropriate injection site can be important because different areas of the body have different blood flow, more or less consistent subcutaneous fat, etc. Poor blood flow or uneven fat distribution near an injection site could lead to inconsistent or slower absorption, potentially affecting the effectiveness of the medication. It can also be important to rotate injection sites to reduce the risk of redness, swelling, discomfort, scarring, etc. In some cases, injectable products are delivered in single-use pens. In some cases, a pen can contain multiple doses and can come with multiple needles. In some cases, a pen may have a fixed dosage. In some cases, a pen can include functionality that allows the user to select a dose, which introduces additional potential for error as a user may select a dose that is too high or too low. This concern can be especially pronounced when users are first starting medication, as there may be a period of time over which the dosage is ramped up. For example, semaglutide for weight loss typically has a ramp up period that lasts for several months. For example, a patient may start with weekly injections of 0.25 mg for four weeks, followed by injections of 0.5 mg for four weeks, followed by weekly injections of 1 mg for four weeks, followed by four or more weeks of weekly injections of 1.7 mg, finally followed by weekly injections of 2.4 mg. Different patients may have somewhat different schedules or may plateau at different dosages. For example, some patients may remain at 1.7 mg per week while others may remain at 2.4 mg per week. Another potential complication is that certain injectable medications may require or benefit from storage under refrigeration but should be warmed to room temperature prior to injection.

Given challenges associated with verifying that medications are authentic and with verifying that medications are administered correctly, there is a need for automated approaches that can verify that medications are authentic and/or that medications are taken correctly.

In some embodiments, a computer system can be configured to access images captured using a high resolution camera or other imaging device. The accessed images can include medication packaging, a medication itself (e.g., a dosing pen, capsule, tablet, liquid, etc.). The system can use computer vision to analyze the images. For example, in some embodiments, the system can identify an object of interest such as a medication, packaging, etc., in the images and extract the object of interest. The extracted object of interest can be compared with one or more reference images to evaluate an authenticity of the medication. In some embodiments, a user can provide a name of the medication, and the extracted object of interest can be compared with images of the named medication. In some embodiments, the system may have prior knowledge of the medication. For example, the user can be a patient who has been prescribed a particular medication as part of a weight loss program. In some embodiments, a user may not be a patient but can instead be a pharmacy, distributor, healthcare facility, etc.

Comparisons can be done in a variety of manners. For example, in some embodiments, the system generates a vector representation of an accessed image and computes a cosine similarity to a vector representation of a reference image. Other vector similarity comparisons, such as L1 distance or L2 distance, are possible. In some embodiments, the system computes a hash of an accessed image, such as a perceptual hash, difference hash, Haar wavelet hash, color hash (e.g., HSV color hash), average hash, etc. The hash can be compared with a hash of a reference image. If the hashes are similar, this can indicate that the medication is authentic. Vector similarity, hash similarity, etc., can be carried out on full images or using extracted objects of interest.

In some embodiments, a system can be configured to look at specific features, such as a QR code, barcode, holographic seal, or other security features. In some embodiments, images can be captured using a high resolution camera, smartphone camera, etc. In some embodiments, medications can be tracked over time, e.g., as they make their way from a manufacturer or distributor to a pharmacy, patient, etc. In some embodiments, tracking information can be stored in a write-only database, on a blockchain, etc., to ensure that nefarious actors are not able to modify records to mask counterfeiting. In some embodiments, users can submit images via a website, smartphone application, etc. In some applications, digital watermarking can be used to track images to ensure, for example, that images are not reused and/or to aid in tracking a medication. For example, a watermark can be applied via least significant bit substitution or noise injection.

As described above, in some embodiments, a system can use cosine similarity to determine medication authenticity. Cosine similarity involves measuring the similarity between two vectors in a vector space. In some embodiments, images or objects extracted from images, can be represented as vectors (e.g., embeddings). Each dimension of a vector can correspond to a specific feature or characteristic. To determine similarity (e.g., similarity between an accessed image and a reference image), the cosine similarity can be calculated. For example, for an accessed image represented by a vector \vec{A} and a reference image represented by a vector \vec{B}, the cosine similarity can be defined as \vec{A}·\vec{B}/(∥\vec{A}∥∥\vec{B}∥). The cosine similarity can range from −1 to 1, with 1 indicating identical vectors, 0 indicating no similarity (e.g., the vectors are orthogonal), and −1 representing complete dissimilarity. In some embodiments, negative values may not be possible, and the cosine similarity can range from 0 to 1.

In some embodiments, a system can determine a match if the cosine similarity meets or exceeds a threshold value. Setting the cosine similarity threshold too high or too low can result in false negatives or false positives. For example, if the cosine similarity is set to 1, only exact matches will be found, leading to false negatives as even a slight difference would prevent matching. On the other hand, setting the cosine similarity threshold too low can result in false positives, in which counterfeit medications are identified as authentic. In some embodiments, a system can enforce an upper bound on similarity. For example, if a cosine similarity is 1, this can indicate that an individual is attempting to pass off a reference image as a legitimate image of a medication.

In some embodiments, the platform can set similarity thresholds based on risk. For example, a greater similarity may be required for the platform to identify a match for a medication that is particularly likely to be counterfeited, such as medications that are expensive, identified as in short supply, or that have high potential for abuse.

In some embodiments, the system can compute one or more hashes for each received image. A hash can include, for example, a perception hash, average hash, wavelet hash, color hash, and/or difference hash. Other hashes are possible. In some embodiments, a hash such as a perception hash can be advantageous because, for example, similar images can be expected to have similar hashes. Thus, hash similarity can be used in some implementations to determine if two images are similar to one another. As described herein, in some embodiments, an object of interest can be extracted from an image. In some embodiments, the platform can be configured to calculate one or more hashes for the object of interest. Similar hashes can indicate authentic medication, while dissimilar hashes may be indicative of counterfeiting.

In some embodiments, a classification model can be trained to classify received image data as indicating authentic or counterfeit medication. Various types of classification or clustering algorithms can be used to identify medications as counterfeit or authentic, such as k-nearest neighbors, support vector machines, and convolutional neural networks. Using k-nearest neighbors (KNN), images can be grouped based on features such that similar images are grouped together. Features can include, for example, pixel values, color distribution, edge patterns, and so forth. When a new image is received, it can be assigned to the most common cluster among its k nearest neighbors. In the case of distinguishing between genuine and counterfeit medications, a KNN algorithm can have k=2 (e.g., a received image is either an image of an authentic medication or a counterfeit medication). In some embodiments, there can be more than two clusters, and the same algorithm can be used to distinguish between genuine and counterfeit medications of different types. Support vector machines can find an optimal hyperplane that separates data points belonging in different classes. Convolutional neural networks are a powerful deep learning architecture that consists of multiple layers of nodes. Each node applies a transformation to its input and provides the resulting output to one or more nodes in the next layer. Convolutional neural networks can offer many advantages because they can learn intricate features automatically from image data, and thus may be well suited to distinguishing between real and counterfeit medications. Convolutional neural networks can be especially advantageous when counterfeits are of high quality and there are only subtle differences between genuine and counterfeit medications. As an example, in some embodiments, a machine learning model can be trained on a data set that includes labeled images of genuine medications and counterfeit medications, and the model can be trained to determine if a newly received image is more similar to the genuine medications or the counterfeit medications.

Images can be captured under a wide variety of conditions and with a wide range of imaging hardware. This can cause an image of a genuine medication to appear different from reference images. Accordingly, in some embodiments, various transformations can be applied prior to analysis, generated vector representations, etc. For example, an image's colors can be normalized or converted to grayscale, contrast and/or brightness can be modified, curves can be modified, resolution can be normalized, and so forth. In some embodiments, a system is configured to adjust images based on image metadata. For example, image metadata may indicate the location and time that an image was captured, which can indicate whether it was sunny or dark. Metadata can include information such as lens, shutter speed, f stop, focal length, resolution, and so forth, which may also indicate imaging conditions. For example, a very slow shutter speed can indicate that an image was captured indoors in poor lighting, while a very fast shutter speed can indicate that an image was captured outside. Imaging conditions and/or metadata can be used for adjusting images, for determining authenticity of an image, or both. For example, if a user is accessing a verification function using an Android device, but an image submitted by the user was captured with an iOS device, it may be likely that an inauthentic image is being passed off. Similarly, timestamps, latitude and longitude, and so forth may reveal that an image was not recently captured or was captured in a different location that where the user is currently located. For example, in some embodiments, a web site or mobile application can be configured to request location information and can use such information to determine where a user is located.

In some embodiments, a system is configured to account for the type of device used to capture an image. For example, the make and model of a smartphone used to capture an image, the operating system version, and so forth can be used in image analysis. For example, the system can store or otherwise have access to information about typical image characteristics for different devices. For example, one brand, series, or model of smartphone may tend to saturate certain colors more than another type of smartphone, or a change in operating system version may include changes to image capture or processing algorithms that affect colors, contrast, sharpness, etc.

As described herein, it can be significant to monitor administration of a medication to ensure that it is administered correctly. While a medication may be authentic, there can be adverse consequences if it is administered incorrectly. This can be especially true when administration is more complex than, for example, swallowing a pill, such as requiring subcutaneous injection at a limited number of injection sites (e.g., the abdomen, thigh, or upper arm). In some embodiments, a user device (e.g., smartphone, tablet, laptop, desktop, headset, etc.) is configured to capture images during an administration process, and computer vision algorithms and/or artificial intelligence/machine learning (AI/ML) models can be configured to monitor the user during administration. For example, a patient can place their smartphone on a stand or otherwise position the smartphone so that a front-facing or rear-facing camera of the smartphone can capture the medication administration process.

One or more algorithms can be configured to identify a patient within an accessed image frame and to identify one or more portions of the patient's body, such as the thighs, abdomen, or upper arm. In some embodiments, the system can identify an injection device such as an injection pen, syringe, and/or the like, determine a distance between the injection device and an acceptable injection location and/or other parts of the patient's body, and alert the patient if they are about to inject into an incorrect area such as the forearm. In some embodiments, the system can cause the patient's device to display an indication that an administration procedure is correct (e.g., that a patient is about to inject into an acceptable site).

In some embodiments, after injection or during injection, a system can be configured to monitor or determine a value of an indicator on the injection device. For example, a syringe can include markers that indicate a level of a plunger in the syringe, or an injection pen can include a visual indicator that indicates when injection is complete or a progress of the injection. In some embodiments, the system can alert the user if the indicator shows that the user did not inject the correct dosage of medication, for example because the user did not fully depress a plunger on a syringe or did not wait long enough for an injection pen to fully inject the medication. In some embodiments, if there is an error during administration, the system can determine next actions (e.g., try again, perform a second injection to make up for a dosage that was too small, skip the dose, consult a physician) and can cause display of instructions on the patient's user device indicating next steps for the patient to take.

In some embodiments, preparatory steps can be monitored additionally or alternatively. For example, prior to injection, a user can be instructed to allow a medication to warm to room temperature for a period of time. A user can place the medication within view of a camera of a user device and the system can monitor an elapsed time and alert the user when sufficient time has elapsed. In some embodiments, a system can monitor the user to ensure that the user disinfects an injection site prior to injection, for example with an alcohol wipe. In some embodiments, the system can monitor the user to ensure that the user correctly installs a needle in an insertion device (e.g., for a reusable pen that contains multiple doses and in which the needle is swapped between injections). In some embodiments, the system can, additionally or alternatively, provide instructions to the user in preparation for administering the medication.

In some embodiments, the approaches herein include timing reminders, for example by maintaining a record of administrations and letting the patient know when it is time for the next dose. In some embodiments, the approaches herein can provide guidance when a patient misses a dose, such as whether or not to take the missed dose, skipped the missed dose, adjust the missed dose, etc.

### Example Environments for Medication Authentication and Administration Verification

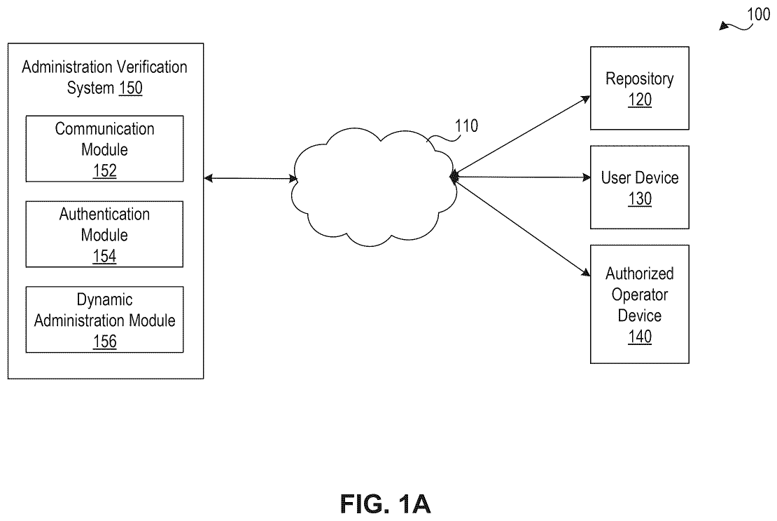

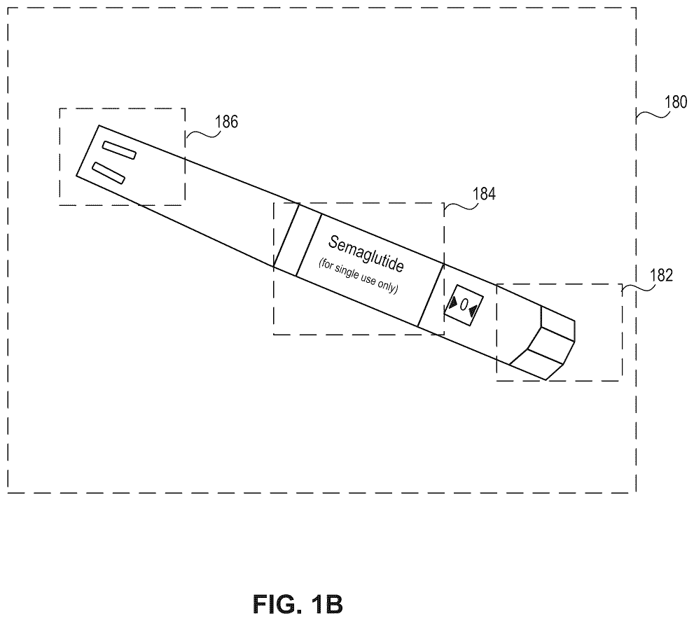

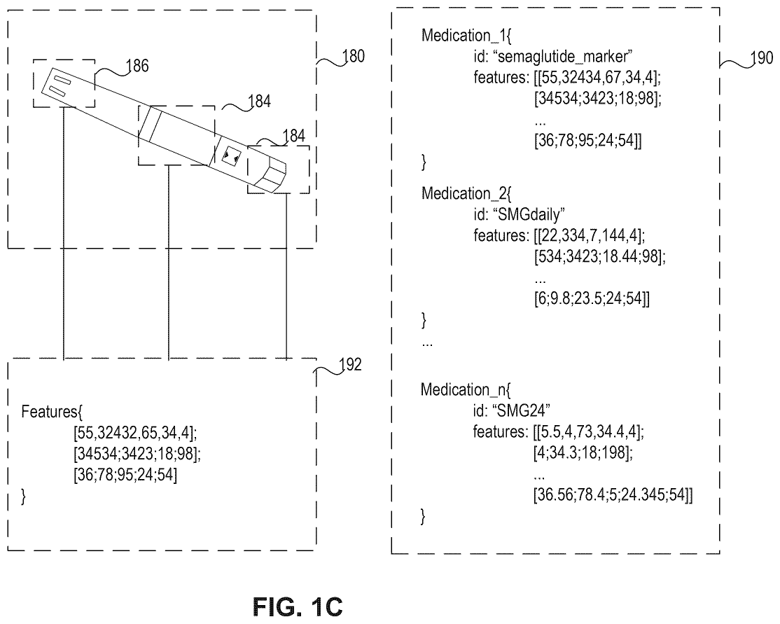

FIG. 1A is an example of an environment 100 for authentication of medications and administration of said medications. Environment 100 can include administration verification system 150, repository 120, user device 130, and authorized operator device 140. Administration verification system 150 may execute instructions for verifying medication, administration of medication, or both, such as according to processes disclosed herein. Administration verification system 150 may include software, hardware, or a combination of the two. For example, administration verification system 150 may be hosted on a physical server or a virtual server that is running on a physical computer system. In some embodiments, administration verification system 150 may be configured on a user device (e.g., a laptop computer, a smartphone, a desktop computer, an electronic tablet, or another suitable user device).

The administration verification system 150 can include functional modules that are implemented with a combination of software (e.g., executable instructions or computer code) and hardware (e.g., at least a memory and processor). Accordingly, as used herein, in some examples a module is a processor-implemented module or set of code and represents a computing device having a processor that is at least temporarily configured and/or programmed by executable instructions stored in memory to perform one or more of the specific functions described herein. For example, the administration verification system 150 includes a communication module 152, authentication module 154, and dynamic administration module 156. The various modules of the administration verification system can operate according to the techniques described herein.

The communication module 152 of the administration verification system 150 can include software and/or hardware components that enable the transmission and/or receipt of information between two or more devices. The communication module 452 can include a wireless communication module, such as a cellular radio or Wi-Fi radio, to allow for communication over wireless networks. In some embodiments, the communications module 152 is a wired network interface card, which can be a separate card or built into a motherboard.

The communication module 152 can be configured and/or programmed (e.g., via the above-mentioned techniques) to interface between a device (e.g., user device 130, authorized user device 140), repositories (e.g., repository 120), and the administration verification system 150, such as via a network (e.g., network 110) to receive requests and images from users for verifying medication, verifying that the user is administering the medication correctly, or both. In some embodiments, the communication module 152 is configured to transmit signals, such as for triggering image capture at a remote device, or for displaying instructions for administering the medication. When the communication module 152 receives data, the module can pass on relevant portions of the data to different modules of the administration verification system 150.

As described herein, communication module 152 of the administration verification system 150 can receive, access, or otherwise obtain, for example, from one or more users at user device 130, a request for verifying administration for a medication. The user at the user device 130 may include a patient, pharmacist, distributor, manufacturer, etc. who wishes to verify the medication or administration of the medication. The user at the user device 130 may include the patient themselves or may include a caregiver, aide, or nurse administering the medication to the patient. The request may be specifically for verifying the medication for the purpose, e.g., prior to administering the medication to the patient, or in the case of a pharmacist or distributor, prior to shipping or packaging the medication for the patient. Alternatively or additionally, the request may be for verifying the process of administering the medication for a patient.

In some embodiments, the request may include at least one image taken of at least a portion of the medication or packaging of the medication. For example, the user may utilize a camera or other imaging sensor of the user device 130 to capture and transmit image data and the request may include an image of medication or packaging taken by the user at the user device 130. In one example, a patient using a GLP-1 receptor agonist injection, such as semaglutide or liraglutide might submit a photo of the injection pen or the box it came in. According to some embodiments, prior to passing the image to the authentication module 154, communication module 152 may evaluate the submitted image against a set of technical quality metrics to ensure it is suitable for medication verification.

For example, the system may assess image resolution (e.g., requiring a minimum of 1024×768 pixels to ensure that fine print, such as dosage or lot numbers, is legible). Similarly, the system may assess sharpness of the image using any of various techniques, such as edge detection algorithms or the variance of the Laplacian, with a predetermined threshold (e.g., variance>100) below which the image is considered blurry. Brightness and contrast can also be measured using histogram analysis. For example, the image pixel values may be compared against acceptable mean pixel intensity values (e.g., between 100 and 200 on a scale of 0-255) to avoid images that are too dark or washed out. The system may, additionally or alternatively, use object detection models to confirm that key regions, such as the entire medication box, the full label, or the injection pen's measurement markings, are present and unobstructed.

If the image fails any of the criteria of the set of technical quality metrics, the communication module 152 can automatically generate a corrective instruction to transmit to the user, e.g., via the network 110, such as “Retake the photo ensuring the entire label is visible and the image is in focus,” or more specifically, “Increase lighting and ensure the resolution is at least 1024×768 pixels.” The corrective instruction may be in an audio or visual format. According to some embodiments, instructions for displaying the corrective instruction may cause the corrective instruction to be superimposed over an image capture display. In some embodiments, a large language model (LLM) may dynamically generate the corrective instructions based on the detected deficiencies, or the system may select from a library of predefined messages tailored to the specific measurement that was not met. Communication module 152 may use the subsequently obtained images to perform authorization and administration tasks, such as responsive to determining that the set of technical quality metrics are met.

According to some embodiments, the set of technical quality metrics used to evaluate submitted images may be specific to a medication or provider, such as based on the requirements specified by the medication's manufacturer or provider. In some embodiments, the technical quality metrics may be predefined and/or static based on guidelines or specifications provided by the manufacturer. In particular, an authorized operator such as at authorized operator device 140 may submit to the administration verification system 150 a set of manufacturer-specific technical quality metrics needed to verify authenticity including values such as minimum resolution, acceptable brightness ranges, or the necessity to capture specific label elements (e.g., dosage, lot number, expiration date). For example, a manufacturer may specify that all images must be at least 1024×768 pixels and that the entire medication box and label must be visible and unobstructed.

Alternatively, the system may dynamically adjust these metrics in response to real-world data or evolving requirements. For instance, if administration verification system 150 detects a pattern of failed verifications due to unreadable lot numbers, administration verification system 150 may automatically increase the minimum sharpness threshold or require higher resolution. Additionally, the system may incorporate feedback from healthcare providers or regulatory bodies (e.g., via the authorized operator device 140) to refine the metrics over time, ensuring ongoing compliance and acceptable image quality.

In response to determining that the image meets each of the technical quality metrics, the communication module 152 may pass the request data, including the image data, or a pointer to the data in memory, to the authentication module 154. The authentication module 154 may be configured to identify regions of interest from the image, each region of interest containing a medication-identifying feature such as machine-readable codes, packaging elements, or tamper-evident features. For example, the authentication module may use a combination of object detection algorithms and optical character recognition (OCR) to isolate and extract these features from the image.

To enable the identification of one or more regions of interest from an image, where each region of interest contains a medication-identifying feature (or other features that can be used for medication authentication, such as brand identifiers), the administration verification system 150 can leverage a computer vision techniques, machine learning techniques, or both. In some embodiments, the system employs one or more object detection algorithms, such as those based on convolutional neural networks (CNNs) like YOLO (You Only Look Once) or Faster R-CNN, to scan the image and localize areas likely to contain relevant features. For example, the model can be trained to recognize the shape and layout of medication packages, the contours of injection pens, or the presence of labels. Once these candidate regions are identified, the system can further analyze them to detect specific medication-identifying features. For instance, a machine-readable code (such as a QR code or barcode) can be detected using specialized barcode detection algorithms, while tamper-evident features (e.g., holographic seals or breakable tabs) can be identified using pattern recognition algorithms, texture analysis algorithms, etc.

FIG. 1B shows an example of an image that depicts an injection pen and various regions that can be detected according to some embodiments of the present disclosure. In the example of FIG. 1B, the image 180 comprises a semaglutide injection pen having regions of interest including region of interest 182, region of interest 184, and region of interest 186. The region of interest 184 may include a medication-identifying feature such as a label, from which text can be identified (e.g., using optical character recognition (OCR) as described herein). In some embodiments, text color, formatting, font, kerning, size, etc., can be used as medication-identifying features. The region of interest 182 may include medication-identifying features of ridges at the base of the medication, whereas the region of interest 186 may include medication-identifying features such as indentations in the distal end of the injection pen.

After isolating the regions of interest, the system can apply OCR technologies to extract textual information from the identified regions, including the medication name, dosage, lot number, expiration date, etc. For example, the system may first use object detection to locate the label area on a medication box, then apply OCR to read the printed text. In the case of machine-readable codes, the system can decode the information to cross-reference it with a database of authorized medications. For tamper-evident features, the system may use image classification models trained on examples of intact versus compromised seals to determine authenticity.

Once the medication-identifying features are extracted, the authentication module 154 may then compare these features to reference features stored in a reference repository, such as repository 120. This reference repository is maintained and updated by authorized operator devices (e.g., authorized operator device 140), which may include devices operated by medication manufacturers, licensed medication providers, regulatory agencies, or other trusted entities. These operator devices are permissioned, e.g., authenticated and granted specific access rights by an entity maintaining the repository to update the repository. For each unique medication identifier, the authorized operator device 140 may submit data to the repository for storage. For example, the data may include a set of medication-identifying features, such as high-resolution images of genuine packaging, expected barcode or QR code values, and descriptions or images of tamper-evident elements. Additionally or alternatively, the authorization module 154 may store feature representations of each medication-identifying feature as disclosed herein.

In some instances, the repository may also include features, or feature representations, of known counterfeit or fake medications, such as images of fraudulent packaging or compromised barcodes. By comparing the extracted features from the user-submitted image to both legitimate and counterfeit entries in the repository, the authentication module 154 can robustly determine the authenticity of the medication. If a match is found with a known fake, the system can flag the medication as potentially counterfeit and trigger transmission of a notification or alert to the user at user device 130. Accordingly, in some embodiments, the systems and methods herein can be used for counterfeit medication attribution and the like, potentially aiding in prosecution of counterfeiters or helping authorities stop counterfeit medications before they make their way to consumers.

As described herein, in some embodiments, in order to conserve computational resources and improve efficiency, the system does not compare the entirety of the medication-identifying features of each region of interest within the image to the features of the repository, but instead generates a feature representation for each. This feature representation may take the form of a hash value (e.g., a cryptographic hash of a barcode or label), a vector embedding (e.g., a numerical vector produced by a neural network trained to encode visual characteristics), or a set of extracted visual features (such as color histograms, edge maps, or texture descriptors).

For example, a hash value can be generated for each medication-identifying feature, such as a barcode or label, and applying a cryptographic hash function (e.g., SHA-356) to the pixel data or decoded content, resulting in a fixed-length string that uniquely represents the input. Alternatively or additionally, the authentication module 154 may generate a vector embedding by passing the portion of the image corresponding to the medication-identifying feature through a neural network (e.g., a convolutional neural network (CNN)) that has been trained to encode visual information, and the network may output a numerical vector in a high-dimensional space, where similar images are mapped to nearby points. Alternatively, or additionally, the authentication module 154 may generate a set of extracted visual features by computing specific descriptors from the image, such as color histograms (which summarize the distribution of colors), edge maps (which highlight boundaries and shapes using algorithms like Canny or Sobel), or texture descriptors (which quantify patterns and surface properties using methods like Local Binary Patterns or Gabor filters). Each of these representations serves to distill the complex visual content of an image into a form that is more suitable for analysis, comparison, or indexing by computer systems. By reducing the image to these compact and information-rich representations, the system can perform rapid and scalable comparisons against a large database of reference features.

The feature representations stored in the repository may also take the form of hashes, vector embeddings, etc. as described herein, such that when comparison occurs, the system can efficiently match the feature representation generated from the region of interest in the input image to the corresponding feature representations stored in the repository. This matching process may involve, for example, comparing hash values for exact matches, calculating similarity scores between vector embeddings using distance metrics (such as cosine similarity, Euclidean distance, or Manhattan distance), or evaluating the degree of correspondence between sets of extracted visual features.

The authorization module 154 may identify, based on the comparing, a matching medication associated with a medication providing entity. As described herein, to identify a match for a medication, the system can extract a set of distinguishing features from the input and transform the features into one or more feature vectors, which may subsequently be compared against precomputed feature vectors stored in the repository for known medications. For each medication entered in the repository, the authorization module 154 or the repository 120 may calculate similarity scores between the input feature representations (e.g., from the request image) and each feature representation for features of the medication of the repository.

FIG. 1C conceptually illustrates feature extraction and comparison to records in a repository according to some embodiments of the present disclosure. FIG. 1C illustrates a repository 190 storing precomputed feature representations for reference medication-identifying features, in accordance with embodiments herein. Each entry of the repository may store data structures such as for “medication_1”, “medication_2”, and “medication_n.” Each data structure may include an identifier for the medication. The identifier may include any unique alphanumeric string that can be used to uniquely identify the medication at the system or the repository. Each data structure may further include precomputed representations for the medications, represented in FIG. 1C as vector embeddings. The authorization module 154 may generate a request or query to the repository to identify whether there is a matching medication and whether the medication is authentic. In the example of FIG. 1A, the authentication module 154 generates feature representations 192, which can be transmitted as part of a query to the repository. The repository 190 may identify that the feature representations for the input image are most similar (e.g., highest similarity measure) to an entry for “medication_1” and may return the values of the entry or may transmit an indication of a match if the similarity exceeds a predetermined threshold or the dissimilarity does not exceed a predetermined threshold. In cases where multiple candidates meet or closely approach the threshold, the system may prompt the user to provide additional data, such as by prompting the user to capture more images or input additional details (e.g., brand name, etc.), to further disambiguate the results.

The system may also determine corresponding administration instructions. As referred to herein, administration instructions may include administration instructions that refer to official, authoritative guidelines or protocols for the proper administration of a medication, which are generated, approved, or distributed by a recognized entity. In some embodiments, the authorized operator device 140 may be permissioned to enter the instructions as part of the repository 190 (e.g., within each data structure corresponding to a medication). Such entities may include regulatory agencies (e.g., the U.S. Food and Drug Administration), pharmaceutical companies, healthcare institutions, or other credentialed organizations responsible for ensuring medication safety and efficacy. The instructions may specify details such as dosage, injection sites, route of administration, timing, preparation steps, contraindications, and any special handling or monitoring requirements. The instructions may include sequential steps, e.g., “remove the medication from the packaging”, “let the medication sit at room temperature for 30 minutes”, “prepare the injection site”, etc. and may additionally, or alternatively, include non-sequential steps such as contextual information (e.g., including warnings, storage requirements, or patient-specific considerations). The system may identify contextual information separately from sequential steps such as using a machine learning model, or based on tags or structure of the instructions.

In the case that the administration instructions are stored within the repository, the repository may transmit, and the communication module 152 may be configured to receive, the administration instructions automatically. Alternatively, or additionally, the administration verification system 150 may generate and transmit a separate search query for retrieving administration instructions from a remote database, wherein the search query comprises the identifier of the medication (e.g., once identified based on a match). Responsive to the search query, the communication module 152 may receive, from the repository, the set of administration instructions.

Alternatively, or additionally, the repository may not store administration instructions for the medication. The system may interpolate, based on identifying instructions from medications having the closest feature values or feature representation values, the instructions. The system may also, instead, perform web-scraping to generate the administration instructions. For example, the system may identify a URL associated with the medication providing entity and generate a request to scrape the data at the URL wherein the request comprises an identifier of the medication. Responsive to receiving scraped data from the URL, the dynamic administration module 156 may generate a set of administration instructions based on the scraped data. In some embodiments, the system can determine administration instructions by analyzing one or more images of the medication or packaging captured by a patient using a user device such as a smartphone. For example, the packaging of the medication can include administration instructions regarding timing, dosage, etc.

As a specific example, dynamic administration module 156 may generate and transmit a web-scraping request to the identified URL, embedding a unique identifier for the medication (e.g., National Drug Code, product name, or other standardized identifier) within the request parameters. Once the communication module 152 receives, as a result of the web-scraping process, the raw data from the specified URL, the dynamic administration module 156 may process the scraped content to extract and structure the relevant administration instructions.

For example, the dynamic administration module 156 may parse HTML, PDF, plain text, Markdown, or other document formats, identifying key sections using natural language processing (NLP) or pattern-matching algorithms (e.g., identifying heading tags in HTML or Markdown), and filtering out extraneous or irrelevant information. The module may compile the extracted data into a standardized set of administration instructions. In some embodiments, the generated set of administration instructions may be transmitted to an authorized operator device for approval, and the instructions may be subsequently stored in the repository within an entry for the corresponding medication. In some embodiments, the generated instructions may be used to provide administration instructions to the user but may be identified as a generated instruction set by displaying a warning or notification to the user. In particular, the communication module 152 may generate commands for superimposing the warning or notification to the display of the user device 130.

If the user's request is solely to verify the medication, the system may simply transmit the administration instructions to the user device 130 for review, for example, as on-screen text or a downloadable file. However, if the request also involves verifying the medication administration process, the system may initiate an image capture workflow on the remote user device (e.g., user device 130) to document or further confirm the administration event, as described further herein.

The dynamic administration module 156 may be configured to verify an administration process of the medication. In particular, the module 156 may be configured to use real-time image data and medication-specific administration instructions to provide instructions to the user (e.g., caregiver or patient) such that the medication is administered properly. In particular, once a matching medication is determined and instructions for administering the medication are retrieved, the system may trigger an image acquisition session at the remote device to obtain a time-series of images, at least one of the images comprising at least a portion of a patient in proximity to the medication. For example, the communication module 152 may, responsive to receiving an indication of a match from the repository, or responsive to identifying a match at the system, may automatically generate and transmit one or more commands configured to trigger an image acquisition session at the user device 130.

For example, the user device 130 may execute an application configured to begin an image acquisition session responsive to a command from the administration verification system 150 (or the application can enable a user or patient to begin an imagine acquisition session on demand). The user device 130 may be configured to turn on a camera function on the device, and display to the user the camera feed. The user device 130 may also be configured to display a message indicating that imaging is being performed (e.g., “Please wait, we are taking some images”). In some embodiments, the user device may be configured to initialize an image acquisition session but may not begin image acquisition until receiving further authorization via a user interaction at the user device 130 to do so, such as receiving a voice command or touch input.

The system may help the user administer the medication correctly, such as by finding the right injection spots, using a correct trajectory (e.g., 45 degree angle, etc.), and/or the like. Furthermore, the administration instructions may include one or more alternatives for administering based on patient-specific variables. For example, if the patient looks to be pregnant or is known to be pregnant based on medical information provided by the patient or accessed from an electronic medical record system, the administration instructions may be different than for non-pregnant patients. Thus, once communication module 152 receives one or more images, the dynamic administration module 156 can extract, based on the time-series of images, data such as one or more patient features and a posture of the medication in relation to the patient.

For example, dynamic administration module 156 may be configured to identify features such as body size, weight, age indicators, skin colors and conditions (e.g., pallor, rashes, jaundice), mobility or physical disability, presence of medical devices, such as insulin pumps, swelling, pregnancy, injection track marks, bruising, tattoos, etc. For example, an estimated weight of a person may affect dosage, particularly for weight-based drugs. Similarly, age indicators may help identify if a patient is elderly, which may require adjusted dosing due to metabolism or comorbidities. Mobility or physical disability may impact the administration method, e.g., oral versus injectable. Medical devices such as oxygen cannulas, IV lines, insulin pumps, etc., may influence the route of administration or indicate existing drug regimens. As another example of circumstance-specific modification, pregnancy may require adjusted dosing. Swollen limbs or face may suggest fluid retention. Dermatological markers such as injection track marks, bruising, tattoos, etc. may impact IV drug use, bleeding disorders, or where not to inject.

In some embodiments, to improve safety and accuracy, the system may employ advanced feature extraction techniques to assess vascularity at the intended injection site. Computer vision algorithms can process the image frames to detect visible blood vessels using color segmentation (e.g., identifying blue or green hues under the skin), vessel pattern recognition, and contrast enhancement. These vascular features can be extracted and used to generate vector embeddings or other feature representations and compared to reference patterns in a centrally managed database to assess the density and location of superficial vessels, for example.

The system can further extract patient-specific vascularity features, such as the presence, size, and branching pattern of veins near the injection site. For example, a convolutional neural network (CNN) trained on annotated vascular datasets may be used to segment and map the vasculature in the region of interest. If prominent veins are detected near the intended injection site, the system may recommend an alternative site or provide a warning to avoid intravascular injection. In some embodiments, vessel depth may be estimated using multi-angle imaging or photoplethysmography (PPG) techniques, further improving injection safety.

The dynamic administration module 156 may also identify, as described herein, a posture of the medication in relation to the patient. For example, the directionality of the medication (e.g., the direction of the needle portion), angle (e.g., of the needle to the skin), trajectory (e.g., directionality of the movement of the injection), and in some cases, velocity, may be identified. In some cases, the user may additionally interact with the user device 130 to mark and indicate an intended injection site (e.g., on the image). The system may determine, based on the image and marking, what portion of the body the intended injection site is selected (e.g., using computer vision techniques or other machine learning).

The dynamic administration module 156 may input the one or more patient features, the posture of the medication in relation to the patient, and administration instructions into a machine learning model to obtain a set of patient-specific administration instructions to direct a user to administer the medication. Additionally, the module may input general guidelines specific to the type of medication. For example, for medications of the type “injections,” the module may include instructions as context, such as to avoid bruised areas when injecting. Additionally, or alternatively, the module may include the user-identified intended injection site.

The model may be trained, e.g., to output instructions based on the identified data. For example, the model may be a large language model. In particular, the dynamic administration module 156 may generate a prompt given the patient features, the administration instructions, and the images (or description of the images, e.g., in the form of the posture). The prompt may be input into the LLM executing locally or remotely (e.g., via communication module 152). The LLM may return, as output, one or more patient-specific administration instructions. For example, the administration instructions may include instructions to update the posture, to change the injection site, to change the dosage, etc. In particular, the system may identify, based on the given data, which of the alternative instruction sets to provide to the user, and how to adjust the user's behavior or actions to better adhere to the identified instruction set. Alternatively or additionally, the administration instructions may include a warning to the user to seek medical attention or cease using the medication (e.g., if the patient is determined to be a child or pregnant). The system may transmit, via communication module 152 to the user device, one or more commands for displaying the set of patient-specific administration instructions at the user device.

In some cases, the system may trigger a second image acquisition session at the remote device to obtain a second time-series of images after receiving the first time-series. The system may determine, based on the second time-series of images, a trajectory of the medication towards the patient and responsive to determining that the trajectory is inconsistent with a predefined administration path, generating and transmitting, to the remote device, a corrective instruction.

In some embodiments, the process may be recorded and transmitted to the authorized operator device 140 such that the medication providers or manufacturers can modify or update the administration instructions based on reviewing the process as implemented by patients and/or caregivers. In some embodiments, prior to transmitting the images and/or video to the authorized operator device 140, the system may perform filtering to remove or mosaic portions of the image identified to include the patient's face, sensitive areas, or identifiable areas (e.g., tattoos, etc.).

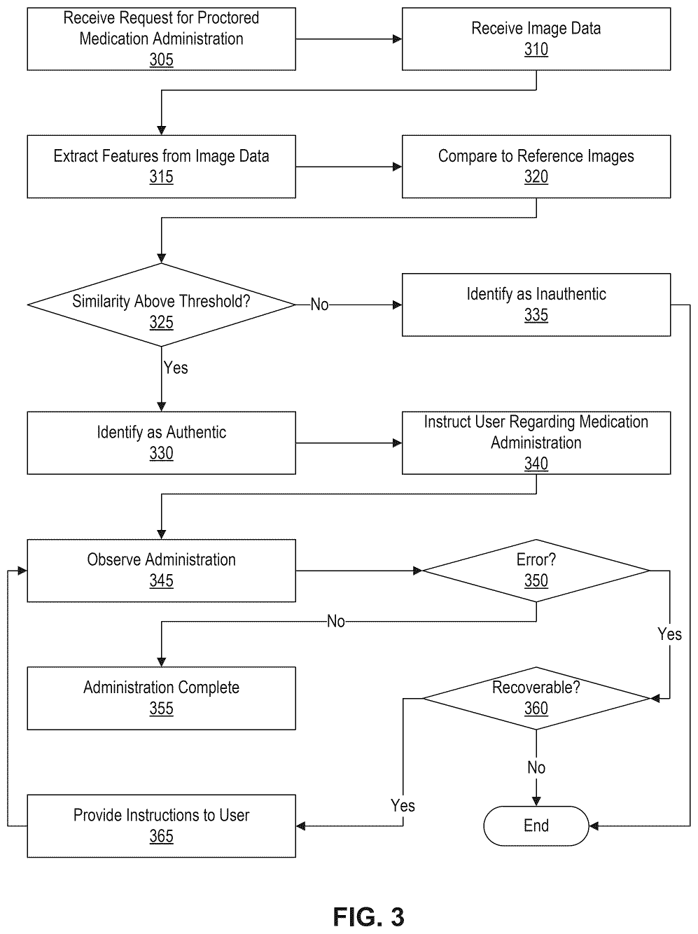

The example embodiments described below in relation to FIG. 3-8 describe example processes that may be performed using the administration verification system 150 described herein. The examples may also be implemented in any other suitable system which may include fewer, additional, or different modules, without departing from the scope of the present disclosure.

### Example Embodiments

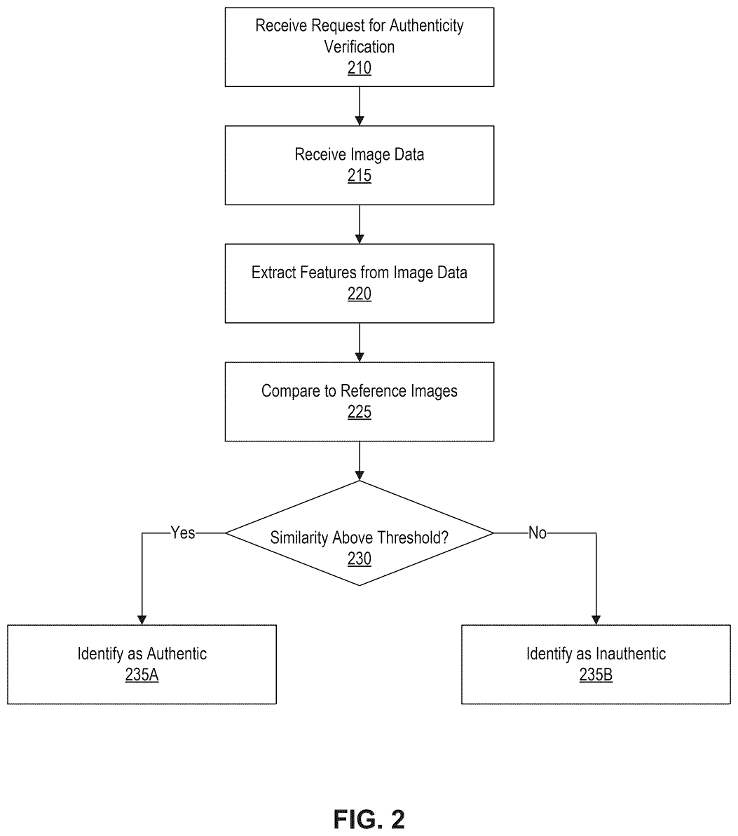

FIG. 2 is a block diagram that illustrates an example process for verifying medication authenticity according to some implementations. At operation 210, a system can receive a request for authenticity verification. As described herein, the system can receive a request from a patient, pharmacist, distributor, manufacturer, etc. At operation 215, the system can receive image data. The image data can include, for example, a still image or video. In the case of video, the system can extract one or more frames from the video for further analysis. At operation 220, the system can extract features from the image data. For example, in some embodiments, the system can extract an object of interest from the image data. In some cases, the extracted object of interest can comprise the entire image (e.g., operation 220 may not be performed).

At operation 225, the system can compare the object of interest to one or more reference images. As described herein, the comparison can be carried out in various ways, such as computing and comparing image hashes, computing and comparing vectorized representations of image data (e.g., using cosine similarity), and classifying image data using a classification model, etc. At operation 230, the system can determine if the similarity is above a threshold value. If so, the system can, at operation 235A, identify the medication as authentic. If not, the system can, at operation 235B, identify the medication as not authentic.

In some embodiments, the system may misidentify a medication. For example, the system can identify an authentic medication as a counterfeit medication. In some cases, the system can prompt a user to submit an additional image, and the process of FIG. 2 can be carried out on the additional image. In some embodiments, a result may not be clear. For example, in some embodiments, a medication may be identified as authentic if a similarity score (e.g., cosine similarity or other vector similarity, or hash similarity) is above a first threshold amount, and may be identified as counterfeit if the similarity score is below a second, different threshold amount. Between the first and second thresholds, the system may output an indeterminate result, in which case the system can prompt the user to submit an additional image and/or the image can be routed to a human for manual review. If the indeterminate result is later determined to be genuine or counterfeit, this information can be used to retrain a classification model or other machine learning model that can be used to distinguish between authentic and counterfeit medications.

FIG. 3 is a block diagram that illustrates an example process for medication authenticity verification and administration verification according to some implementations. At operation 305, a computer system can receive a request for proctored medication administration. In some embodiments, a user accesses proctored medication administration after providing login credentials (e.g., a username and password or the like). At operation 310, the system can receive image data. The image data can show a medication to be administered. At operation 315, the system can extract features from the image data. At operation 320, the system can compare to extracted features (or the received image data as a whole) to one or more reference images, for example by computing vector similarity, hash similarity, using a machine learning model such as a convolutional neural network, etc. At operation 325, if the similarity is below a threshold, the system can identify the medication as inauthentic at operation 335 and the process can stop. In some embodiments, the system can prompt the user to provide additional image data (e.g., to capture another image) so that authentication can be attempted again.

If, at operation 325, the similarity is above the threshold, the system can identify the medication as authentic at operation 330. At operation 340, the system can provide instructions to the user regarding administration of the medication, such as information about preparation (e.g., disinfecting an injection site, allowing the medication to warm to room temperature, etc.), administration of the medication, etc. At operation 345, the system can observe administration of the medication, for example via a video feed from a user device. In some embodiments, the system can extract frames from the video feed or can process the video feed without extracting frames. At operation 350, if the system does not detect an error during the process, administration can be complete at operation 355. If the system does detect an error at operation 350, the system can determine if the error is recoverable at operation 360. For example, if a user does not wait long enough for the medication to warm before injection, the system can prompt the user to wait longer, or if the user places an injection device at an incorrect location, the system can prompt the user to move the injection device prior to proceeding. In some cases, an error may not be recoverable, such as if the user depresses a plunger or injection button before a needle is inserted into the user. If, at operation 360, the error is not recoverable, the process can end. If, at operation 360, the error is recoverable, the system can provide instructions to the user (e.g., wait longer before injecting, change injection sites, etc.) at operation 365, and the process can continue with the system continuing to observe administration at operation 345.

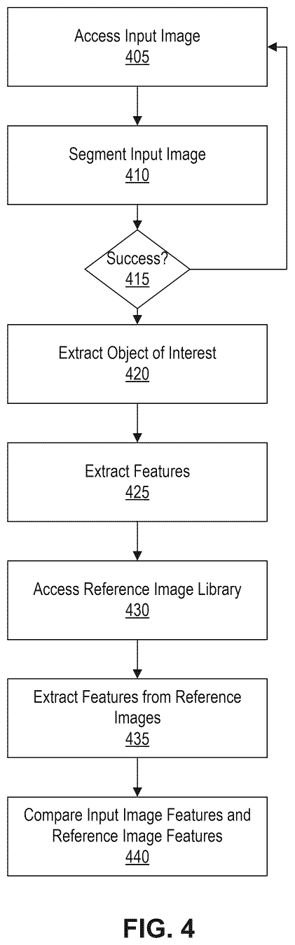

FIG. 4 is a flowchart that illustrates an example process for comparing an input image to images in a reference library according to some implementations. At operation 405, a system can access an input image, for example as captured by a camera of a user device. At operation 410, the system can segment the input image, for example using an object detection algorithm such as YOLO or Faster R-CNN. In some embodiments, at operation 410, the system generates a bounding box or segmentation mask around the detected object (e.g., medication packaging). At operation 415, if the segmentation was unsuccessful, the system can access another input image. For example, the system can prompt a user to capture a second image. If segmentation was successful, the system can extract the object at operation 420, for example by cropping or masking the input image. In some embodiments, extracting the object generates a new image. At operation 425, the system can extract features from the object, which can be compared to features of reference images. Feature extraction can include, for example, extracting a color histogram, analyzing texture, edge detection, determining shapes (e.g., aspect ratios, circularity, object distribution, etc.), and so forth. In some embodiments, the system using a convolutional neural network to extract features that can capture intricate details and relationships in the image.

At operation 430, the system can access references images in a reference image library and can, at operation 435, extract features from the reference images. In some cases, features may be extracted ahead of time and stored so that operations 430 and 435 do not need to be carried out for each received input image.

At operation 440, the system can compare the features of the extracted object from the input image to features of one or more reference images in the reference image library using a similarity metric, such as Euclidean distance, cosine similarity, or Jaccard similarity. The comparison can be used to identify whether the input image depicts an object that is authentic or counterfeit. For example, in some embodiments, the reference image library can include images of authentic medications, and the system can determine that the medication in the input image is authentic if there is a match with at least a threshold confidence. In some embodiments, the reference library can, additionally or alternatively, include images of counterfeit medications. If the input image shows a medication that is similar to a counterfeit medication (e.g., more than a threshold similarity), the system can determine that the input image depicts a counterfeit medication.

In some cases, medication packaging includes machine-readable codes such as barcodes, QR codes, etc. Machine-readable codes can be specific to a particular package or can be used for multiple packages. For example, a bar code or other machine-readable code can be a code that generally can be used to identify a medication or can be a code that allows identification of the specific package. Machine-readable codes can also be used for other levels of granularity, such as identifying a manufacturing location, lot number, etc. Machine-readable codes are not strictly limited to barcodes, QR codes, and the like, but can be any information on the medication or packaging that can be read by a computer, such as printed text. In some embodiments, a system can be configured to use a machine-readable code to verify a medication or as part of a medication verification.

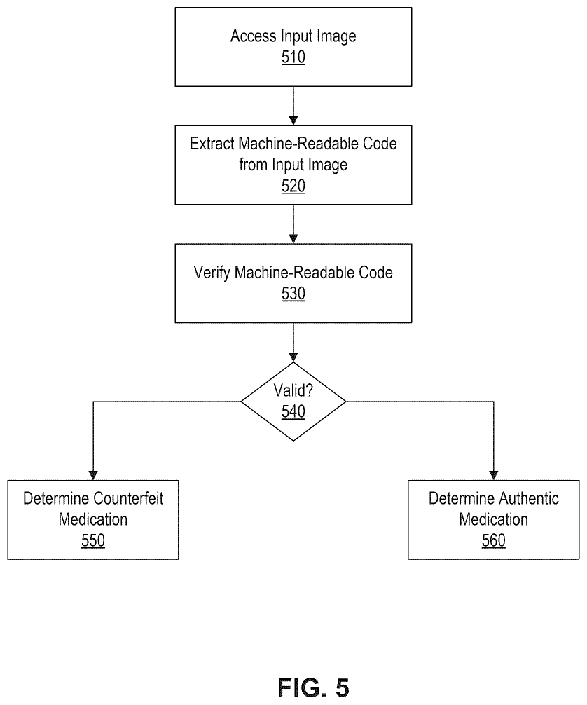

FIG. 5 is a flowchart that illustrates an example process for verifying a medication using a machine-readable code according to some implementations. At operation 510, a system can access an input image, for example an image of a medication or medication package submitted by a patient. At operation 520, the system can extract a machine-readable code from the input image. At operation 530, the system can verify the machine-readable code. Verifying the machine-readable code can be accomplished in various ways. For example, in some embodiments, a pharmaceutical company makes available functionality that can be used to check the machine-readable code against a database of valid information. In some embodiments, another service, such as a telehealth service or virtual weight loss program, can operate a database that stores information about valid medications, and the machine-readable code can be checked against said database. At operation 540, if the machine-readable code indicates is unable to be verified, the system can determine that the medication is counterfeit at operation 550. If the machine-readable code indicates that the machine-readable code is valid, the system can determine that the medication is authentic at operation 560.

As described herein, medication package can include tamper-evident features. These tamper-evident features may indicate if a medication has been tampered with during transit, or can be used to identify counterfeit products. For example, a counterfeit product may attempt to use the same holographic sticker, security tape, shrink wrap, tamper-evident closures (e.g., caps or lids), and so forth. However, often there are differences in the tamper-evident features on genuine medications and counterfeit medications. In some cases, counterfeiters may attempt to closely replicate the tamper-evident features used for a genuine medication. In other cases, counterfeiters may add tamper-evident features to provide the appearance of authenticity, but the tamper-evident features may be significantly different from the features of genuine products. This can be effective because, for example, patients, pharmacists, etc., are likely unaware of what the tamper-evident features are supposed to look like, exactly which tamper-evident features should be present, etc. Moreover, manufacturers may change such features from time to time, so even if a patient, pharmacist, etc., is familiar with the tamper-evident features of a genuine product, they may not be suspicious if a product appears that has different tamper-evident features.

Various techniques can be used to detect if tamper-evident packaging is consistent with genuine medications, if a medication has been tampered with, and so forth. For example, if different tamper-evident features are used, a system can determine that a product is counterfeit. If tamper-evident features show signs of tampering, the system can determine that a medication may have been tampered with. For example, there may be dust or other residue present if a stick or tape has been lifted and replaced, features may be misaligned, seals may appear broken or defective, shrinkwrap may show certain defects or differences, and so forth.

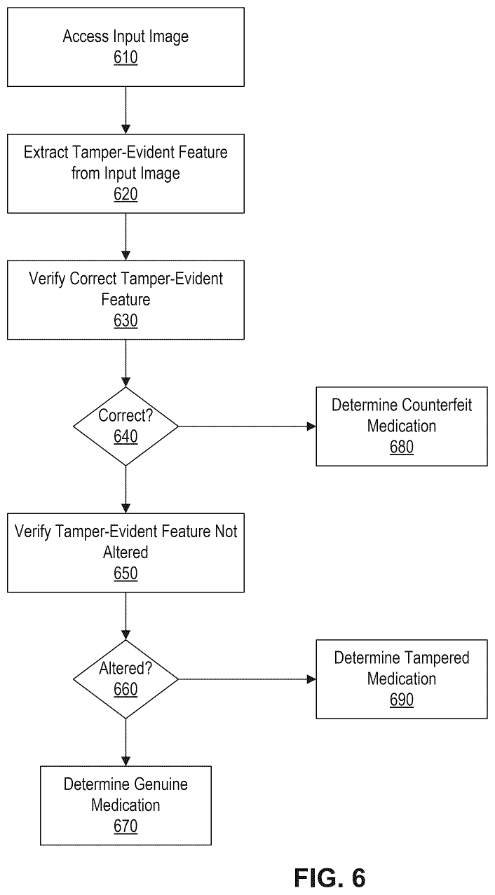

FIG. 6 is a flowchart that illustrates an example process for analyzing tamper-evident features according to some implementations. At operation 610, a system can access an input image, for example an image of a medication package. At operation 620, the system can extract a portion of the image showing a tamper-evident feature, for example using a computer vision or machine learning algorithm. At operation 630, the system can verify that the tamper-evident feature is a correct tamper-evident feature, for example by computing a similarity between the extracted tamper-evident feature and reference images of tamper-evident features. The approaches described herein with respect to other comparisons (e.g., vector similarity, hash similarity, CNN computations, etc.) can be used for verifying the tamper-evident feature. At operation 640, the system can determine if the tamper-evident feature is consistent with a genuine tamper-evident feature. If not, the system can determine that the medication is a counterfeit medication at operation 680. If so, the system can analyze the tamper-evident feature to determine if the tamper-evident feature shows signs of tampering, such as peeling, dust or debris, misalignment, etc. At operation 660, if the tamper-evident feature shows signs of alteration, the system can determine that the medication has been tampered with at operation 690. If not, the system can determine that the medication shown in the input image is a genuine medication at operation 670.

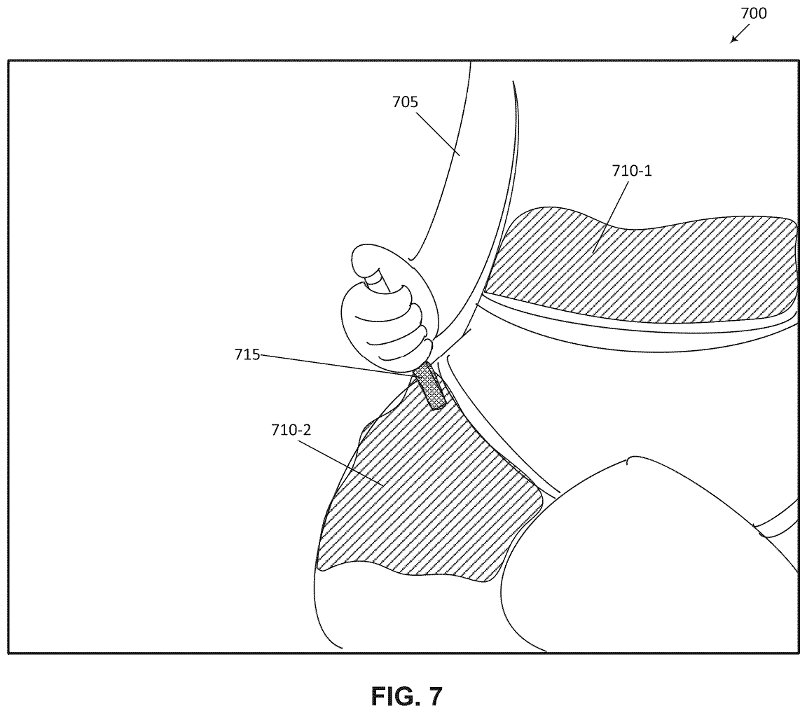

FIG. 7 is a diagram that illustrates an example of identifying injection sites and an injection device according to some implementations. In FIG. 7, an image frame 700 depicts a patient 705. A computer-vision algorithm and/or machine learning algorithm can be configured to identify injection sites, which can vary depending upon the medication being administered. For example, two injection sites 710-1 (abdomen) and 710-2 (thigh) are identified in FIG. 7. These injection sites can be suitable for a subcutaneous injection. A system can, additionally or alternatively, detect an injection device 715, such as a syringe or injection pen. In some embodiments, the system can measure a distance between an injection site and an injection device, an angle between an injection site and an injection device, and so forth. If the injection device is placed in an appropriate position for injection into an injection site, the system can cause an alert or message to be displayed on a user device of the patient indicating proper placement of the injection device. If the injection device is not properly placed, the system can be configured to cause a message to be displayed on the user device indicating that the injection device is not properly positioned.

In some embodiments, the system may utilize a sequence of image frames to monitor the trajectory of the injection device relative to the identified injection site(s). For example, after detecting both the injection device and an injection site, the system can trigger a second image acquisition session to capture a time-series of images as the user moves the injection device toward the patient. By analyzing the spatial and temporal changes in the position and orientation of the injection device, the system can determine whether the device is following a predefined administration path, such as a straight-line approach at a specified angle (e.g., 90 degrees for subcutaneous injection). If the trajectory deviates from the expected path, such as approaching at an oblique angle or targeting an incorrect site, the system can generate and transmit a corrective instruction to the user device, such as “Adjust angle to 90°” or “Move device closer to the abdomen.”

Feedback can be provided to the user during the injection process based on vascularity analysis. As the injection device approaches the site, the system may display a visual overlay on the user device highlighting detected vessels and indicating safe zones for injection. If the device is aligned with a region of high vascularity, the system can issue a prompt such as “Move device to avoid visible vein” or “Select alternative site.” Upon successful placement in a low-vascularity area, the system may display a confirmation message, such as “Safe to inject,” and optionally trigger a countdown timer to guide the user through the injection duration as specified by the instructions.

Prior to administration, the system can verify the medication by analyzing machine-readable codes (e.g., QR codes, barcodes), tamper-evident features present on the medication packaging, and so forth. The extracted code or feature representation can be compared to a repository of valid codes, images of features of genuine medications, images of features of counterfeit medications, and so forth, to establish authenticity and retrieve the corresponding administration instructions. For example, upon scanning a QR code on a prefilled injection pen, the system may retrieve the manufacturer's recommended injection sites, dosage, and administration technique, and display these instructions to the user in real time.

Relevant data from the verification and administration process, such as timestamps, device and site positions, vascularity maps, and any corrective instructions issued, may be securely transmitted to a remote server for audit, compliance, or integration with electronic health records (EHRs). The system can generate a summary report for the patient or healthcare provider, documenting successful administration, failed administration, any deviations or interventions that occurred during the process, and so forth. In some embodiments, certain functions can be carried out on the user device itself. That is, in some embodiments, a system and a user device can be the same device.

### Machine Learning

A “model,” as used herein, can refer to a construct that is trained using training data to make predictions or provide probabilities for new data items, whether or not the new data items were included in the training data. For example, training data for supervised learning can include items with various parameters and an assigned classification. A new data item can have parameters that a model can use to assign a classification to the new data item. As another example, a model can be a probability distribution resulting from the analysis of training data, such as a likelihood of an n-gram occurring in a given language based on an analysis of a large corpus from that language. Examples of models include neural networks, support vector machines, decision trees, Parzen windows, Bayes, clustering, reinforcement learning, probability distributions, decision trees, decision tree forests, and others. Models can be configured for various situations, data types, sources, and output formats.

In some embodiments, a model can be a neural network with multiple input nodes that receive features extracted from an image, which can be a still image, a frame extracted from a video or video feed, etc. The input nodes can correspond to functions that receive the input and produce results. These results can be provided to one or more levels of intermediate nodes that each produce further results based on a combination of lower-level node results. A weighting factor can be applied to the output of each node before the result is passed to the next layer node. At a final layer, (“the output layer”) one or more nodes can produce a value classifying the input that, once the model is trained, can be used for various functions, such as to determine if a medication is genuine or counterfeit. In some embodiments, such neural networks, can have multiple layers of intermediate nodes with different configurations, can be a combination of models that receive different parts of the input and/or input from other parts of the deep neural network, or are convolutions-partially using output from previous iterations of applying the model as further input to produce results for the current input.

A machine learning model can be trained with supervised learning, where the training data includes images as input and a desired output, such as a classification as genuine or counterfeit. A representation of the images (e.g., extracted features) can be provided to the model. Output from the model can be compared to the desired output for a particular image and, based on the comparison, the model can be modified, such as by changing weights between nodes of the neural network or parameters of the functions used at each node in the neural network (e.g., applying a loss function). After applying each of the images in the training data and modifying the model in this manner, the model can be trained to evaluate new images.

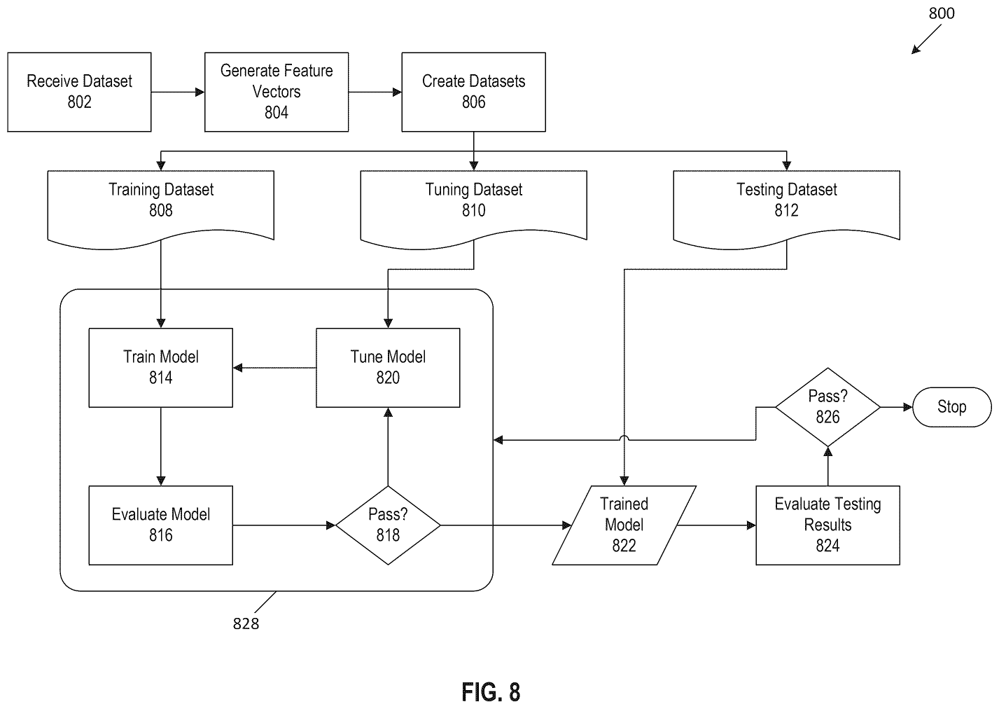

FIG. 8 depicts a process for training an artificial intelligence or machine learning model according to some embodiments. The process 800 can be run on a computing system. At block 802, the system may access or receive a dataset. The dataset can include images of genuine medications, counterfeit medications, or both. In some embodiments, the images can be classified or labeled. For example, image sample may be classified as showing a genuine medication or a counterfeit medication. At block 804, the images can be used to generate feature vectors. In some embodiments, various transformations can be applied to the images prior to generating feature vectors. For example, images may require transformations to conform to expected input formats, for example to conform with expected resolution, color space, image format, etc. These are merely examples, and the skilled artisan will readily appreciate that other transformations are possible. At block 806, the system may create, from the received dataset, training, tuning, and testing/validation datasets. The training dataset 808 may be used during training to determine features for forming a predictive model. The tuning dataset 810 may be used to select final models and to prevent or correct overfitting that may occur during training with the training dataset 808, as the trained model should be generally applicable to a broad range of input data, not merely to data used for model training. The testing dataset 812 may be used after training and tuning to evaluate the model. For example, the testing dataset 812 may be used to check if the model is overfitted to the training dataset. The system, in training loop 828, may train the model at block 814 using the training dataset 808. Training may be conducted in a supervised, unsupervised, or partially supervised manner. According to various embodiments herein, models can be trained in a supervised manner. At 816, the system may evaluate the model according to one or more evaluation criteria. For example, the evaluation may include determining how often a model correctly or incorrectly identifies a counterfeit or genuine medication. At decision point 809, the system may determine if the model meets the one or more evaluation criteria. If the model fails evaluation, the system may, at 820, tune the model using the tuning dataset 810, repeating the training loop 828 and evaluation 816 until the model passes the evaluation at decision point 818. Once the model passes the evaluation at 809, the system may exit the model training loop 828. The testing dataset 812 may be run through the trained model 822 and, at block 824, the system may evaluate the results. If the evaluation fails, at block 826, the system may reenter training loop 828 for additional training and tuning. If the model passes, the system may stop the training process, resulting in a trained model 822. In some embodiments, the training process may be modified. For example, the system may not use a tuning dataset 810. In some embodiments, the model may not use a testing dataset 812.

### Example Clauses

Embodiment 1. A system for remote verification of medication administration, the system comprising: one or more processors; and one or more memories configured to store instructions that when executed by the one or more processors cause the system to: receive, from a remote device, a request for verifying administration for a medication, the request comprising at least one image; identify one or more regions of interest from the at least one image, wherein the one or more regions of interest each comprise a medication identifying feature; compare a feature representation of each medication identifying feature to a plurality of reference feature representations corresponding to one or more medications and/or medication packaging, wherein each reference feature representation is centrally managed by one or more authorized operator devices; identify, based on the comparing, a matching medication associated with a medication providing entity and corresponding administration instructions; trigger an image acquisition session at the remote device to obtain a time-series of images, at least one of the images comprising at least a portion of a patient in proximity to the medication; extract, based on the time-series of images, (i) one or more patient features and (ii) a posture of the medication in relation to the patient; input the one or more patient features, the posture of the medication in relation to the patient, and administration instructions into a machine learning model to obtain a set of patient-specific administration instructions to direct a user to administer the medication; and transmit, to the remote device, one or more commands for displaying the set of patient-specific administration instructions.

Embodiment 2. The system of embodiment 1, wherein the medication identifying feature comprises at least one of: a medication, an injection device, a medication package, a machine-readable code, or a tamper-evident feature.

Embodiment 3. The system of embodiment 1, wherein the instructions further cause the one or more processors to perform operations comprising: triggering a second image acquisition session at the remote device to obtain a second time-series of images; determining, based on the second time-series of images, a trajectory of the medication towards the patient; responsive to determining that the trajectory is inconsistent with a predefined administration path, generating and transmitting, to the remote device, a corrective instruction.

Embodiment 4. The system of embodiment 1, wherein the feature representation comprises at least one of: a hash value, a vector embedding, or a set of extracted visual features.

Embodiment 5. The system of embodiment 1, wherein the instructions for identifying the corresponding administration instructions further cause the system to: generate a search query for retrieving administration instructions from a remote database, wherein the query comprises an identifier of the medication; transmit the query to the remote data; and receive, from the remote database, a set administration instructions.

Embodiment 6. The system of embodiment 1, wherein the instructions for identifying the corresponding entity-issued administration instructions further cause the one system to: identify a URL associated with the medication providing entity; generate a request to scrape the data at the URL wherein the request comprises an identifier of the medication; receive scraped data from the URL; and generate a set of administration instructions based on the scraped data.

Embodiment 7. A method for remote verification of medication administration, the method comprising: receiving, from a remote device, a request for verifying administration for a medication, the request comprising at least one image; identifying one or more regions of interest from the at least one image, wherein the one or more regions of interest each comprise a medication identifying feature; comparing a feature representation of each medication identifying feature to a plurality of reference feature representations corresponding to one or more medications and/or medication packaging, wherein each reference feature representation is centrally managed by one or more authorized operator devices; identifying, based on the comparing, a matching medication associated with a medication providing entity and corresponding administration instructions; triggering an image acquisition session at the remote device to obtain a time-series of images, at least one of the images comprising at least a portion of a patient in proximity to the medication; extracting, based on the time-series of images, (i) one or more patient features and (ii) a posture of the medication in relation to the patient; inputting the one or more patient features, the posture of the medication in relation to the patient, and administration instructions into a machine learning model to obtain a set of patient-specific administration instructions to direct a user to administer the medication; and transmitting, to the remote device, one or more commands for displaying the set of patient-specific administration instructions.

Embodiment 8. The method of embodiment 7, wherein the medication identifying feature comprises at least one of: a medication, an injection device, a medication package, a machine-readable code, or a tamper-evident feature.

Embodiment 9. The method of embodiment 7, further comprising: triggering a second image acquisition session at the remote device to obtain a second time-series of images; determining, based on the second time-series of images, a trajectory of the medication towards the patient; responsive to determining that the trajectory is inconsistent with a predefined administration path, generating and transmitting, to the remote device, a corrective instruction.

Embodiment 10. The method of embodiment 7, wherein the feature representation comprises at least one of: a hash value, a vector embedding, or a set of extracted visual features.

Embodiment 11. The method of embodiment 7, wherein identifying the corresponding administration instructions comprises: generating a search query for retrieving administration instructions from a remote database, wherein the search query comprises an identifier of the medication; transmitting the search query to the remote database; and receive, from the remote database, a set of administration instructions.

Embodiment 12. The method of embodiment 7, wherein identifying the corresponding administration instructions comprises: identifying a URL associated with the medication providing entity; generating a request to scrape the data at the URL wherein the request comprises an identifier of the medication; receiving scraped data from the URL; and generating a set of administration instructions based on the scraped data.

Embodiment 13. One or more non-transitory, computer-readable media storing instructions thereon, where the instructions when executed by one or more processors cause operations comprising: receiving, from a remote device, a request for verifying administration for a medication, the request comprising at least one image; identifying one or more regions of interest from the at least one image, wherein the one or more regions of interest each comprise a medication identifying feature; comparing a feature representation of each medication identifying feature to a plurality of reference feature representations corresponding to one or more medications and/or medication packaging; identifying, based on the comparing, a matching medication associated with a medication providing entity and corresponding administration instructions; and transmitting one or more commands for triggering an image acquisition session at the remote device to obtain a time-series of images, at least one of the images comprising at least a portion of a patient in proximity to the medication.

Embodiment 14. The one or more non-transitory, computer-readable media of embodiment 13, wherein the operations further comprise: extracting, based on the time-series of images, (i) one or more patient features and (ii) a posture of the medication in relation to the patient; inputting the one or more patient features, the posture of the medication in relation to the patient, and administration instructions into a machine learning model to obtain a set of patient-specific administration instructions to direct a user to administer the medication; and transmitting, to the remote device, one or more commands for displaying the set of patient-specific administration instructions.

Embodiment 15. The one or more non-transitory, computer-readable media of embodiment 14, wherein the medication identifying feature comprises at least one of: a medication, a medication package, a machine-readable code, or a tamper-evident feature.

Embodiment 16. The one or more non-transitory, computer-readable media of embodiment 14, wherein the operations further comprise: determining an authenticity of the image, wherein determining the authenticity comprises analyzing one or more of: a capture time, a shutter speed, a location, a user device model of a remote device used for capturing the at least one image, a make of the remote device, a focal length, a resolution, a lens, or an f-stop.

Embodiment 17. The one or more non-transitory, computer-readable media of embodiment 14, wherein the feature representation comprises at least one of: a hash value, a vector embedding, or a set of extracted visual features.

Embodiment 18. The one or more non-transitory, computer-readable media of embodiment 14, wherein the operations for identifying the corresponding administration instructions further comprise: generating and transmitting a search query for retrieving administration instructions from a remote database, wherein the search query comprises an identifier of the medication; and receive, from the remote database, a set of administration instructions.

Embodiment 19. The one or more non-transitory, computer-readable media of embodiment 14, wherein the operations for identifying the corresponding administration instructions further comprise: identifying a URL associated with the medication providing entity; generating a request to scrape the data at the URL wherein the request comprises an identifier of the medication; receiving scraped data from the URL; and generating a set of administration instructions based on the scraped data.

Embodiment 20. The one or more non-transitory, computer-readable media of embodiment 13, wherein each reference feature representation is centrally managed by one or more authorized operator devices.

### Computer System

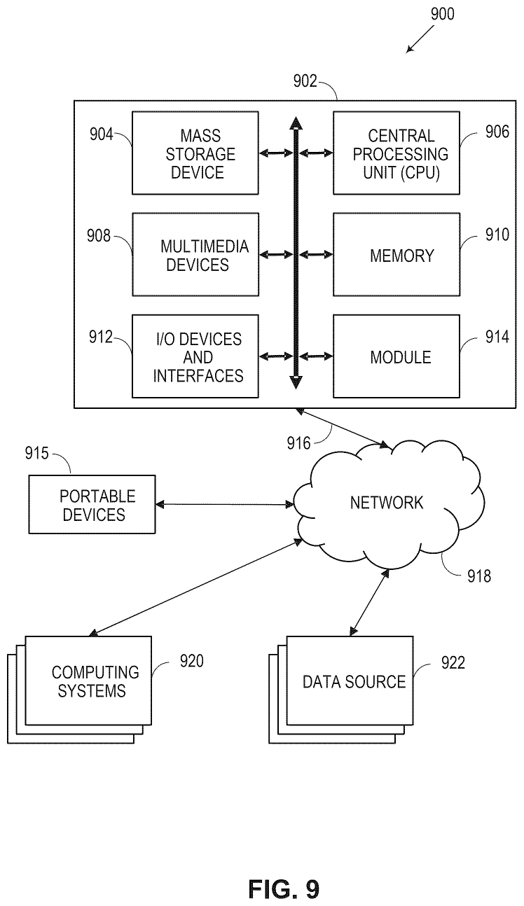

FIG. 9 is a block diagram 900 depicting an embodiment of a computer hardware system 902 configured to run software for implementing one or more of the systems and methods described herein. The example computer system 902 is in communication with one or more computing systems 920 and/or one or more data sources 922 via one or more networks 918. While FIG. 9 illustrates an embodiment of a computing system 902, it is recognized that the functionality provided for in the components and modules of computer system 902 may be combined into fewer components and modules, or further separated into additional components and modules.

The computer system 902 can comprise a module 914 that carries out the functions, methods, acts, and/or processes described herein. The module 914 is executed on the computer system 902 by a central processing unit 906 discussed further below.

In general, the word “module,” as used herein, refers to logic embodied in hardware or firmware or to a collection of software instructions, having entry and exit points. Modules are written in a program language, such as JAVA, C or C++, PYTHON, or the like. Software modules may be compiled or linked into an executable program, installed in a dynamic link library, or may be written in an interpreted language such as BASIC, PERL, LUA, or PYTHON. Software modules may be called from other modules or from themselves, and/or may be invoked in response to detected events or interruptions. Modules implemented in hardware include connected logic units such as gates and flip-flops, and/or may include programmable units, such as programmable gate arrays or processors.

Generally, the modules described herein refer to logical modules that may be combined with other modules or divided into sub-modules despite their physical organization or storage. The modules are executed by one or more computing systems and may be stored on or within any suitable computer readable medium or implemented in-whole or in-part within special designed hardware or firmware. Not all calculations, analysis, and/or optimization require the use of computer systems, though any of the above-described methods, calculations, processes, or analyses may be facilitated through the use of computers. Further, in some embodiments, process blocks described herein may be altered, rearranged, combined, and/or omitted.

The computer system 902 includes one or more processing units (CPU) 906, which may comprise a microprocessor. The computer system 902 further includes a physical memory 910, such as random-access memory (RAM) for temporary storage of information, a read only memory (ROM) for permanent storage of information, and a mass storage device 904, such as a backing store, hard drive, rotating magnetic disks, solid state disks (SSD), flash memory, phase-change memory (PCM), 3D XPoint memory, diskette, or optical media storage device. Alternatively, the mass storage device may be implemented in an array of servers. Typically, the components of the computer system 902 are connected to the computer using a standards-based bus system. The bus system can be implemented using various protocols, such as Peripheral Component Interconnect (PCI), Micro Channel, SCSI, Industrial Standard Architecture (ISA) and Extended ISA (EISA) architectures.

The computer system 902 includes one or more input/output (I/O) devices and interfaces 912, such as a keyboard, mouse, touch pad, and printer. The I/O devices and interfaces 912 can include one or more display devices, such as a monitor, which allows the visual presentation of data to a user. More particularly, a display device provides for the presentation of GUIs as application software data, and multi-media presentations, for example. The I/O devices and interfaces 912 can also provide a communications interface to various external devices. The computer system 902 may comprise one or more multi-media devices 908, such as speakers, video cards, graphics accelerators, and microphones, for example.

The computer system 902 may run on a variety of computing devices, such as a server, a Windows server, a Structure Query Language server, a Unix Server, a personal computer, a laptop computer, and so forth. In other embodiments, the computer system 902 may run on a cluster computer system, a mainframe computer system and/or other computing system suitable for controlling and/or communicating with large databases, performing high volume transaction processing, and generating reports from large databases. The computing system 902 is generally controlled and coordinated by an operating system software, such as z/OS, Windows, Linux, UNIX, BSD, SunOS, Solaris, MacOS, or other compatible operating systems, including proprietary operating systems. Operating systems control and schedule computer processes for execution, perform memory management, provide file system, networking, and I/O services, and provide a user interface, such as a graphical user interface (GUI), among other things.

The computer system 902 illustrated in FIG. 9 is coupled to a network 918, such as a LAN, WAN, or the Internet via a communication link 916 (wired, wireless, or a combination thereof). Network 918 communicates with various computing devices and/or other electronic devices, such as portable devices 915. Network 918 is communicating with one or more computing systems 920 and one or more data sources 922. The module 914 may access or may be accessed by computing systems 920 and/or data sources 922 through a web-enabled user access point. Connections may be a direct physical connection, a virtual connection, and other connection type. The web-enabled user access point may comprise a browser module that uses text, graphics, audio, video, and other media to present data and to allow interaction with data via the network 918.

Access to the module 914 of the computer system 902 by computing systems 920 and/or by data sources 922 may be through a web-enabled user access point such as the computing systems' 920 or data source's 922 personal computer, cellular phone, smartphone, laptop, tablet computer, e-reader device, audio player, or another device capable of connecting to the network 918. Such a device may have a browser module that is implemented as a module that uses text, graphics, audio, video, and other media to present data and to allow interaction with data via the network 918.

The output module may be implemented as a combination of an all-points addressable display such as a cathode ray tube (CRT), a liquid crystal display (LCD), a plasma display, or other types and/or combinations of displays. The output module may be implemented to communicate with interfaces 912 and they also include software with the appropriate interfaces which allow a user to access data through the use of stylized screen elements, such as menus, windows, dialogue boxes, tool bars, and controls (for example, radio buttons, check boxes, sliding scales, and so forth). Furthermore, the output module may communicate with a set of input and output devices to receive signals from the user.

The input device(s) may comprise a keyboard, roller ball, pen and stylus, mouse, trackball, voice recognition system, or pre-designated switches or buttons. The output device(s) may comprise a speaker, a display screen, a printer, or a voice synthesizer. In addition, a touch screen may act as a hybrid input/output device. In another embodiment, a user may interact with the system more directly such as through a system terminal connected to the score generator without communications over the Internet, a WAN, or LAN, or similar network.

In some embodiments, the system 902 may comprise a physical or logical connection established between a remote microprocessor and a mainframe host computer for the express purpose of uploading, downloading, or viewing interactive data and databases on-line in real time. The remote microprocessor may be operated by an entity operating the computer system 902, including the client server systems or the main server system, an/or may be operated by one or more of the data sources 922 and/or one or more of the computing systems 920. In some embodiments, terminal emulation software may be used on the microprocessor for participating in the micro-mainframe link.

In some embodiments, computing systems 920 who are internal to an entity operating the computer system 902 may access the module 914 internally as an application or process run by the CPU 906.

In some embodiments, one or more features of the systems, methods, and devices described herein can utilize a URL and/or cookies, for example for storing and/or transmitting data or user information. A Uniform Resource Locator (URL) can include a web address and/or a reference to a web resource that is stored on a database and/or a server. The URL can specify the location of the resource on a computer and/or a computer network. The URL can include a mechanism to retrieve the network resource. The source of the network resource can receive a URL, identify the location of the web resource, and transmit the web resource back to the requestor. A URL can be converted to an IP address, and a Domain Name System (DNS) can look up the URL and its corresponding IP address. URLs can be references to web pages, file transfers, emails, database accesses, and other applications. The URLs can include a sequence of characters that identify a path, domain name, a file extension, a host name, a query, a fragment, scheme, a protocol identifier, a port number, a username, a password, a flag, an object, a resource name and/or the like. The systems disclosed herein can generate, receive, transmit, apply, parse, serialize, render, and/or perform an action on a URL.

A cookie, also referred to as an HTTP cookie, a web cookie, an internet cookie, and a browser cookie, can include data sent from a website and/or stored on a user's computer. This data can be stored by a user's web browser while the user is browsing. The cookies can include useful information for websites to remember prior browsing information, such as a shopping cart on an online store, clicking of buttons, login information, and/or records of web pages or network resources visited in the past. Cookies can also include information that the user enters, such as names, addresses, passwords, credit card information, etc. Cookies can also perform computer functions. For example, authentication cookies can be used by applications (for example, a web browser) to identify whether the user is already logged in (for example, to a web site). The cookie data can be encrypted to provide security for the creator. Tracking cookies can be used to compile historical browsing histories of individuals. Systems disclosed herein can generate and use cookies to access data of an individual. Systems can also generate and use JSON web tokens to store authenticity information, HTTP authentication as authentication protocols, IP addresses to track session or identity information, URLs, and the like.

The computing system 902 may include one or more internal and/or external data sources (for example, data sources 922). In some embodiments, one or more of the data repositories and the data sources described above may be implemented using a relational database, such as DB2, Sybase, Oracle, CodeBase, and Microsoft® SQL Server as well as other types of databases such as a flat-file database, an entity relationship database, and object-oriented database, and/or a record-based database.

The computer system 902 may also access one or more databases 922. The databases 922 may be stored in a database or data repository. The computer system 902 may access the one or more databases 922 through a network 918 or may directly access the database or data repository through I/O devices and interfaces 912. The data repository storing the one or more databases 922 may reside within the computer system 902.

### Conclusion

In the foregoing specification, the systems and processes have been described with reference to specific embodiments thereof. It will, however, be evident that various modifications and changes may be made thereto without departing from the broader spirit and scope of the embodiments disclosed herein. The specification and drawings are, accordingly, to be regarded in an illustrative rather than restrictive sense.

Indeed, although the systems and processes have been disclosed in the context of certain embodiments and examples, it will be understood by those skilled in the art that the various embodiments of the systems and processes extend beyond the specifically disclosed embodiments to other alternative embodiments and/or uses of the systems and processes and obvious modifications and equivalents thereof. In addition, while several variations of the embodiments of the systems and processes have been shown and described in detail, other modifications, which are within the scope of this disclosure, will be readily apparent to those of skill in the art based upon this disclosure. It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes of the embodiments of the disclosed systems and processes. Any methods disclosed herein need not be performed in the order recited. Thus, it is intended that the scope of the systems and processes herein disclosed should not be limited by the particular embodiments described above.

It will be appreciated that the systems and methods of the disclosure each have several innovative aspects, no single one of which is solely responsible or required for the desirable attributes disclosed herein. The various features and processes described above may be used independently of one another or may be combined in various ways. All possible combinations and sub-combinations are intended to fall within the scope of this disclosure.

Certain features that are described in this specification in the context of separate embodiments also may be implemented in combination in a single embodiment. Conversely, various features that are described in the context of a single embodiment also may be implemented in multiple embodiments separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination. No single feature or group of features is necessary or indispensable to each and every embodiment.

It will also be appreciated that conditional language used herein, such as, among others, “can,” “could,” “might,” “may,” “for example,” and the like, unless specifically stated otherwise, or otherwise understood within the context as used, is generally intended to convey that certain embodiments include, while other embodiments do not include, certain features, elements and/or operations. Thus, such conditional language is not generally intended to imply that features, elements and/or operations are in any way required for one or more embodiments or that one or more embodiments necessarily include logic for deciding, with or without author input or prompting, whether these features, elements and/or operations are included or are to be performed in any particular embodiment. The terms “comprising,” “including,” “having,” and the like are synonymous and are used inclusively, in an open-ended fashion, and do not exclude additional elements, features, acts, operations, and so forth. In addition, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise. Similarly, while operations may be depicted in the drawings in a particular order, it is to be recognized that such operations need not be performed in the particular order shown or in sequential order, or that all illustrated operations be performed, to achieve desirable results. Further, the drawings may schematically depict one or more example processes in the form of a flowchart. However, other operations that are not depicted may be incorporated in the example methods and processes that are schematically illustrated. For example, one or more additional operations may be performed before, after, simultaneously, or between any of the illustrated operations. Additionally, the operations may be rearranged or reordered in other embodiments. In certain circumstances, multitasking and parallel processing may be advantageous. Moreover, the separation of various system components in the embodiments described above should not be understood as requiring such separation in all embodiments, and it should be understood that the described program components and systems may generally be integrated together in a single software product or packaged into multiple software products. Additionally, other embodiments are within the scope of the following claims. In some cases, the actions recited in the claims may be performed in a different order and still achieve desirable results.

Further, while the methods and devices described herein may be susceptible to various modifications and alternative forms, specific examples thereof have been shown in the drawings and are herein described in detail. It should be understood, however, that the embodiments are not to be limited to the particular forms or methods disclosed, but, to the contrary, the embodiments are to cover all modifications, equivalents, and alternatives falling within the spirit and scope of the various implementations described and the appended claims. Further, the disclosure herein of any particular feature, aspect, method, property, characteristic, quality, attribute, element, or the like in connection with an implementation or embodiment can be used in all other implementations or embodiments set forth herein. Any methods disclosed herein need not be performed in the order recited. The methods disclosed herein may include certain actions taken by a practitioner; however, the methods can also include any third-party instruction of those actions, either expressly or by implication. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (for example, as accurate as reasonably possible under the circumstances, for example ±5%, ±10%, ±15%, etc.). For example, “about 8.5 mm” includes “3.5 mm.” Phrases preceded by a term such as “substantially” include the recited phrase and should be interpreted based on the circumstances (for example, as much as reasonably possible under the circumstances). For example, “substantially constant” includes “constant.” Unless stated otherwise, all measurements are at standard conditions including temperature and pressure.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiments require at least one of X, at least one of Y, and at least one of Z to each be present. The headings provided herein, if any, are for convenience only and do not necessarily affect the scope or meaning of the devices and methods disclosed herein.

Accordingly, the claims are not intended to be limited to the embodiments shown herein but are to be accorded the widest scope consistent with this disclosure, the principles and the novel features disclosed herein.

What is claimed is:

1. A system for remote verification of medication administration, the system comprising:
one or more processors; and
one or more memories configured to store instructions that when executed by the one or more processors cause the system to:
receive, from a remote device, a request for verifying administration for a medication, the request comprising at least one image;
identify one or more regions of interest from the at least one image, wherein the one or more regions of interest each comprise a medication identifying feature;
compare a feature representation of each medication identifying feature to a plurality of reference feature representations corresponding to one or more medications and/or medication packaging, wherein each reference feature representation is centrally managed by one or more authorized operator devices;
identify, based on the comparing, a matching medication associated with a medication providing entity and corresponding administration instructions;
trigger an image acquisition session at the remote device to obtain a time-series of images, at least one of the images comprising at least a portion of a patient in proximity to the medication;
extract, based on the time-series of images, (i) one or more patient features and (ii) a posture of the medication in relation to the patient;
input the one or more patient features, the posture of the medication in relation to the patient, and administration instructions into a machine learning model to obtain a set of patient-specific administration instructions to direct a user to administer the medication; and
transmit, to the remote device, one or more commands for displaying the set of patient-specific administration instructions.

2. The system of claim 1, wherein the medication identifying feature comprises at least one of: a medication, an injection device, a medication package, a machine-readable code, or a tamper-evident feature.

3. The system of claim 1, wherein the instructions further cause the one or more processors to perform operations comprising:
triggering a second image acquisition session at the remote device to obtain a second time-series of images;
determining, based on the second time-series of images, a trajectory of the medication towards the patient;
responsive to determining that the trajectory is inconsistent with a predefined administration path, generating and transmitting, to the remote device, a corrective instruction.

4. The system of claim 1, wherein the feature representation comprises at least one of: a hash value, a vector embedding, or a set of extracted visual features.

5. The system of claim 1, wherein the instructions for identifying the corresponding administration instructions further cause the system to:
generate a search query for retrieving administration instructions from a remote database, wherein the query comprises an identifier of the medication;
transmit the query to the remote database; and
receive, from the remote database, a set administration instructions.

6. The system of claim 1, wherein the instructions for identifying the corresponding administration instructions further cause the one system to:
identify a URL associated with the medication providing entity;
generate a request to scrape data at the URL, wherein the request comprises an identifier of the medication;
receive scraped data from the URL; and
generate a set of administration instructions based on the scraped data.

7. A method for remote verification of medication administration, the method comprising:
receiving, from a remote device, a request for verifying administration for a medication, the request comprising at least one image;
identifying one or more regions of interest from the at least one image, wherein the one or more regions of interest each comprise a medication identifying feature;
comparing a feature representation of each medication identifying feature to a plurality of reference feature representations corresponding to one or more medications and/or medication packaging, wherein each reference feature representation is centrally managed by one or more authorized operator devices;
identifying, based on the comparing, a matching medication associated with a medication providing entity and corresponding administration instructions;
triggering an image acquisition session at the remote device to obtain a time-series of images, at least one of the images comprising at least a portion of a patient in proximity to the medication;
extracting, based on the time-series of images, (i) one or more patient features and (ii) a posture of the medication in relation to the patient;
inputting the one or more patient features, the posture of the medication in relation to the patient, and administration instructions into a machine learning model to obtain a set of patient-specific administration instructions to direct a user to administer the medication; and
transmitting, to the remote device, one or more commands for displaying the set of patient-specific administration instructions.

8. The method of claim 7, wherein the medication identifying feature comprises at least one of: a medication, an injection device, a medication package, a machine-readable code, or a tamper-evident feature.

9. The method of claim 7, further comprising:
triggering a second image acquisition session at the remote device to obtain a second time-series of images;
determining, based on the second time-series of images, a trajectory of the medication towards the patient;
responsive to determining that the trajectory is inconsistent with a predefined administration path, generating and transmitting, to the remote device, a corrective instruction.

10. The method of claim 7, wherein the feature representation comprises at least one of: a hash value, a vector embedding, or a set of extracted visual features.

11. The method of claim 7, wherein identifying the corresponding administration instructions comprises:
generating a search query for retrieving administration instructions from a remote database, wherein the search query comprises an identifier of the medication;
transmitting the search query to the remote database; and
receive, from the remote database, a set of administration instructions.

12. The method of claim 7, wherein identifying the corresponding administration instructions comprises:
identifying a URL associated with the medication providing entity;
generating a request to scrape data at the URL, wherein the request comprises an identifier of the medication;
receiving scraped data from the URL; and
generating a set of administration instructions based on the scraped data.

13. One or more non-transitory, computer-readable media storing instructions thereon, where the instructions when executed by one or more processors cause operations comprising:
receiving, from a remote device, a request for verifying administration for a medication, the request comprising at least one image;
identifying one or more regions of interest from the at least one image, wherein the one or more regions of interest each comprise a medication identifying feature;
comparing a feature representation of each medication identifying feature to a plurality of reference feature representations corresponding to one or more medications and/or medication packaging;
identifying, based on the comparing, a matching medication associated with a medication providing entity and corresponding administration instructions; and
transmitting one or more commands for triggering an image acquisition session at the remote device to obtain a time-series of images, at least one of the images comprising at least a portion of a patient in proximity to the medication.

14. The one or more non-transitory, computer-readable media of claim 13, wherein the operations further comprise:
extracting, based on the time-series of images, (i) one or more patient features and (ii) a posture of the medication in relation to the patient;
inputting the one or more patient features, the posture of the medication in relation to the patient, and administration instructions into a machine learning model to obtain a set of patient-specific administration instructions to direct a user to administer the medication; and
transmitting, to the remote device, one or more commands for displaying the set of patient-specific administration instructions.

15. The one or more non-transitory, computer-readable media of claim 14, wherein the medication identifying feature comprises at least one of: a medication, a medication package, a machine-readable code, or a tamper-evident feature.

16. The one or more non-transitory, computer-readable media of claim 14, wherein the operations further comprise:
determining an authenticity of the image, wherein determining the authenticity comprises analyzing one or more of: a capture time, a shutter speed, a location, a user device model of a remote device used for capturing the at least one image, a make of the remote device, a focal length, a resolution, a lens, or an f-stop.

17. The one or more non-transitory, computer-readable media of claim 14, wherein the feature representation comprises at least one of: a hash value, a vector embedding, or a set of extracted visual features.

18. The one or more non-transitory, computer-readable media of claim 14, wherein the operations for identifying the corresponding administration instructions further comprise:
generating and transmitting a search query for retrieving administration instructions from a remote database, wherein the search query comprises an identifier of the medication; and
receive, from the remote database, a set of administration instructions.

19. The one or more non-transitory, computer-readable media of claim 14, wherein the operations for identifying the corresponding administration instructions further comprise:
identifying a URL associated with the medication providing entity;
generating a request to scrape data at the URL, wherein the request comprises an identifier of the medication;
receiving scraped data from the URL; and
generating a set of administration instructions based on the scraped data.

20. The one or more non-transitory, computer-readable media of claim 13, wherein each reference feature representation is centrally managed by one or more authorized operator devices.
